SEQUENCE LISTING (40 sequences)

SEQ ID NO: 1 - protein, 729 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 2 - DNA, 3599 bp, Homo sapiens
gcccgttgtctgtgtgtgggactgaggggccccgggggcggtgggggctcccggtgggggcagcggtggggagggagggcctggacatggcgctgaggggccgccccgcgggaagatgaataagggctggctggagctggagagcgacccaggcctcttcaccctgctcgtggaagatttcggtgtcaagggggtgcaagtggaggagatctacgaccttcagagcaaatgtcagggccctgtatatggatttatcttcctgttcaaatggatcgaagagcgccggtcccggcgaaaggtctctaccttggtggatgatacgtccgtgattgatgatgatattgtgaataacatgttctttgcccaccagctgatacccaactcttgtgcaactcatgccttgctgagcgtgctcctgaactgcagcagcgtggacctgggacccaccctgagtcgcatgaaggacttcaccaagggtttcagccctgagagcaaaggatatgcgattggcaatgccccggagttggccaaggcccataatagccatgccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatgggccctggggggaggacgaggagtggacagacaaggcccggcgggtcatcatggagcgtatcggcctcgccactgcaggggagccctaccacgacatccgcttcaacctgatggcagtggtgcccgaccgcaggatcaagtatgaggccaggctgcatgtgctgaaggtgaaccgtcagacagtactagaggctctgcagcagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggaggaagaagacctggcggcaggtgtgggccgcagccgagttccagtccgcccaccccagcagtactcagatgatgaggatgactatgaggatgacgaggaggatgacgtgcagaacaccaactctgcccttaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggagctgctggcactgctgaagtgtgtggaggctgagattgcaaactatgaggcgtgcctcaaggaggaggtagagaagaggaagaagttcaagattgatgaccagagaaggacccacaactacgatgagttcatctgcacctttatctccatgctggctcaggaaggcatgctggccaacctagtggagcagaacatctccgtgcggcggcgccaaggggtcagcatcggccggctccacaagcagcggaagcctgaccggcggaaacgctctcgcccctacaaggccaagcgccagtgaggactgctggccctgactctgcagcccactcttgccgtgtggccctcaccagggtccttccctgccccacttccccttttcccagtattactgaatagtcccagctggagagtccaggccctgggaatgggaggaaccaggccacattccttccatcgtgccctgaggcctgacacggcagatcagccccatagtgctcaggaggcagcatctggagttggggcacagcgaggtactgcagcttcctccacagccggctgtggagcagcaggacctggcccttctgcctgggcagcagaatatatattttacctatcagagacatctatttttctgggctccaacccaacatgccaccatgttgacataagttcctacctgactatgctttctctcctaggagctgtcctggtgggcccaggtccttgtatcatgccacggtcccaactacagggtcctagctgggggcctgggtgggccctgggctctgggccctgctgctctagccccagccaccagcctgtccctgttgtaaggaagccaggtcttctctcttcattcctcttaggagagtgccaaactcagggacccagcactgggctgggttgggagtagggtgtcccagtggggttggggtgagcaggctgctgggatcccatggcctgagcagagcatgtgggaactgttcagtggcctgtgaactgtcttccttgttctagccaggctgttcaagactgctctccatagcaaggttctagggctcttcgccttcagtgttgtggccctagctatgggcctaaattgggctctaggtctctgtccctggcgcttgaggctcagaagagcctctgtccagcccctcagtattaccatgtctccctctcaggggtagcagagacagggttgcttataggaagctggcaccactcagctcttcctgctactccagtttcctcagcctctgcaaggcactcagggtgggggacagcaggatcaagacaacccgttggagcccctgtgttccagaggacctgatgccaaggggtaatgggcccagcagtgcctctggagcccaggccccaacacagccccatggcctctgccagatggctttgaaaaaggtgatccaagcaggcccctttatctgtacatagtgactgagtggggggtgctggcaagtgtggcagctgcctctgggctgagcacagcttgacccctctagcccctgtaaatactggatcaatgaatgaataaaactctcctaagaatctcctgagaaaaaaaaaaaaaaaaa

SEQ ID NO: 3 - DNA, 17 bp, Artificial sequence
  forward primer
ggagggcctggacatgg

SEQ ID NO: 4 - DNA, 17 bp, Artificial sequence
  reverse primer
atgagtgagggcgcagg

SEQ ID NO: 5 - DNA, 19 bp, Artificial sequence
  forward primer
gggctgtccttccctactg

SEQ ID NO: 6 - DNA, 20 bp, Artificial sequence
  reverse primer
cctgttctctgggaccttcc

SEQ ID NO: 7 - DNA, 20 bp, Artificial sequence
  forward primer
attgtcttctcccctttggc

SEQ ID NO: 8 - DNA, 18 bp, Artificial sequence
  reverse primer
aacatggcagcatcccac

SEQ ID NO: 9 - DNA, 20 bp, Artificial sequence
  forward primer
gtgaggggtgctgtgtatgg

SEQ ID NO: 10 - DNA, 18 bp, Artificial sequence
  reverse primer
agttggctgtgagccagg

SEQ ID NO: 11 - DNA, 20 bp, Artificial sequence
  forward primer
tttgccttccacccatagtc

SEQ ID NO: 12 - DNA, 18 bp, Artificial sequence
  reverse primer
actcccaccccacatcag

SEQ ID NO: 13 - DNA, 18 bp, Artificial sequence
  forward primer
gctgatgtggggtgggag

SEQ ID NO: 14 - DNA, 20 bp, Artificial sequence
  reverse primer
ggaggtaggcagagacaccc

SEQ ID NO: 15 - DNA, 20 bp, Artificial sequence
  forward primer
actcagggtttccttctcgc

SEQ ID NO: 16 - DNA, 20 bp, Artificial sequence
  reverse primer
tctgtccctcccaaagtagg

SEQ ID NO: 17 - DNA, 17 bp, Artificial sequence
  forward primer
ctcaacctgatggcggg

SEQ ID NO: 18 - DNA, 17 bp, Artificial sequence
  reverse primer
aatgcagggagggttgg

SEQ ID NO: 19 - DNA, 20 bp, Artificial sequence
  forward primer
cgggtctaccctttctcctc

SEQ ID NO: 20 - DNA, 19 bp, Artificial sequence
  reverse primer
agacattagcgggtggctc

SEQ ID NO: 21 - DNA, 19 bp, Artificial sequence
  forward primer
ggaggtcctgcctgtgttc

SEQ ID NO: 22 - DNA, 20 bp, Artificial sequence
  reverse primer
ggaaccacatgggaaaattg

SEQ ID NO: 23 - DNA, 18 bp, Artificial sequence
  forward primer
ccgagcagcacttgtttg

SEQ ID NO: 24 - DNA, 20 bp, Artificial sequence
  forward primer
gatccgaagcacctagaacc

SEQ ID NO: 25 - DNA, 17 bp, Artificial sequence
  forward primer
gcccgttcccttgcttc

SEQ ID NO: 26 - DNA, 18 bp, Artificial sequence
  reverse primer
gtgagggctgcgagtgtg

SEQ ID NO: 27 - DNA, 22 bp, Artificial sequence
  forward primer
cctctcaattcctctgtccatc

SEQ ID NO: 28 - DNA, 19 bp, Artificial sequence
  reverse primer
gcaggctgtcatcctctcc

SEQ ID NO: 29 - DNA, 18 bp, Artificial sequence
  forward primer
ctatccgctcagccaacc

SEQ ID NO: 30 - DNA, 17 bp, Artificial sequence
  reverse primer
tccctcctccctcctgg

SEQ ID NO: 31 - DNA, 20 bp, Artificial sequence
  forward primer
ccacaaagtgtcctgcactc

SEQ ID NO: 32 - DNA, 20 bp, Artificial sequence
  reverse primer
agctcaggccttaccctctg

SEQ ID NO: 33 - DNA, 17 bp, Artificial sequence
  forward primer
gtggggctttgttgctg

SEQ ID NO: 34 - DNA, 18 bp, Artificial sequence
  reverse primer
cagtggacctcgggagag

SEQ ID NO: 35 - DNA, 20 bp, Artificial sequence
  forward primer
agattggctccagtgctctc

SEQ ID NO: 36 - DNA, 20 bp, Artificial sequence
  reverse primer
agcagggcattccagttaag

SEQ ID NO: 37 - DNA, 20 bp, Artificial sequence
  forward primer
atgagagcctcagctcctgg

SEQ ID NO: 38 - DNA, 17 bp, Artificial sequence
  reverse primer
cacacggcaagagtggg

SEQ ID NO: 39 - DNA, 20 bp, Artificial sequence
  forward primer
atctgggtcctgtcatcagc

SEQ ID NO: 40 - DNA, 20 bp, Artificial sequence
  reverse primer
acggagatgttctgctccac

(19) United States
(12) Patent Application Publication
(10) Pub. No.: US 2014/0011696 A1
(43) Pub. Date: Jan. 9, 2014

(54) BAP1 MUTATIONAL ANALYSIS IN DETERMINING SUSCEPTIBILITY TO, PROGNOSIS OF, AND TREATMENT OF MELANOCYTIC NEOPLASIA

(71) Applicants: Thomas Wiesner, New York, NY (US); Boris Christoph Bastian, Mill Valley, CA (US); Michael R. Speicher, Hart bei Graz (AT); Heinz Kutzner, Friedrichshafen (DE)

(72) Inventors: Thomas Wiesner, New York, NY (US); Boris Christoph Bastian, Mill Valley, CA (US); Michael R. Speicher, Hart bei Graz (AT); Heinz Kutzner, Friedrichshafen (DE)

(21) Appl. No.: 13/985,912

(22) PCT Filed: Feb. 16, 2012
(86) PCT No.: PCT/US12/25509

§ 371 Date: Sep. 24, 2013

Related U.S. Application Data
(60) Provisional application No. 61/463,389, filed on Feb. 16, 2011.

(51) Int. Cl. C12Q 1/68; G01N 33/68

(52) U.S. Cl. CPC C12Q 1/6886; G01N 33/6893
USPC 506/9; 435/7.1

(57) ABSTRACT

Methods and compositions for diagnosing and prognosing neoplasms, particularly malignant melanomas, and for identifying patients at high risk for melanocytic nevi and/or melanomas or other cancers are provided. Methods for distinguishing between nevi at high and low risk for malignant transformation and for characterizing or classifying lesions or nevi are also disclosed. Assays and kits for prognosis of melanoma in a human subject comprising assessment of BAP1 protein and/or BAP1 nucleic acid are provided.

### FIELD OF THE INVENTION

The present invention relates generally to methods and compositions for diagnosing, prognosing and treating neoplasms, particularly melanocytic neoplasms, including benign epithelioid melanocytic nevi and malignant melanomas, and for identifying patients at high risk for melanocytic nevi and/or melanomas or other cancers. The invention provides methods for distinguishing between nevi at high and low risk for malignant transformation and for characterizing or classifying lesions or nevi.

### BACKGROUND OF THE INVENTION

Melanoma, the most serious type of skin cancer, develops in the cells that produce melanin—the pigment that gives your skin its color. Melanoma can also form in your eyes (uveal melanoma) and, rarely, in the mucous membrane that lines the nose, mouth, esophagus, urinary tract, anus, vagina as mucosal melanoma. Subungual melanoma is another rare form that occurs under a nail and can affect the hands or the feet.

The American Cancer Society estimates that there were over 68,000 cases of melanoma diagnosed in the United States in 2010 alone. Approximately 10% of melanomas represent inherited forms of the disease. Conventional approaches to the clinical management of melanocytic neoplasms rely on properly classifying the type of lesion present. Proper classification, in turn, relies primarily on clinical features, including gross appearance and cell morphology. However, analyses at the molecular level reveal that the melanocytic neoplasms defined by conventional classification schemes are, often, quite heterogeneous. Recent efforts to classify melanocytic lesions, including melanomas, have, therefore, focused on identifying the specific genetic abnormalities that drive the growth of specific lesion types. Such genetic abnormalities can then serve as markers of disease and/or as targets for therapy.

Common acquired melanocytic nevi are benign neoplasms that are composed of small, uniform melanocytes and are typically present as flat or slightly elevated pigmented lesions on the skin. A Spitz nevus is a benign melanocytic nevus affecting the epidermis and dermis. Atypical Spitz tumors (ASTs) are an ill-defined and probably heterogenous group of melanocytic tumors that display histologic features seen in both Spitz nevi and melanomas. Methods and approaches for characterizing, distinguishing and monitoring benign versus malignant skin lesions and for identifying individuals or lesions with propensity to or risk of malignancy are needed.

The BRCA1-associated protein-1 gene (ubiquitin carboxy-terminal hydrolase; BAP1) was originally discovered because of the interaction of the protein it encodes with that encoded by the breast cancer 1, early onset gene (BRCA1) (Jensen, et al. (1998) Oncogene 16(9):1097-1112). The nucleic acid and amino acid sequences of BAP1 are known and publicly available (Genbank NM_{—}004656.2, Genbank NP 004647.1, U.S. Pat. No. 6,307,035). BAP1 has been functionally implicated in the DNA damage response (Matsuoka, et al. (2007) Science 316(5828):1160-1166; Stokes, et al. (2007) PNAS 104(50):19855-19860) as well as in the regulation of apoptosis, senescence and the cell cycle (Ventii, et al. (2008) Cancer Research 68(17):6953-6962). The Drosophila counterpart of BAP1, calypso, has been implicated in chromatin remodeling; its ubiquitin carboxy-terminal hydrolase activity opposes the monoubiquitination activity of the polycomb repressive complex 1, a critical component of transcriptional silencing (Scheuermann, et al. (2010) Nature 465(7925):243-247).

Deletions and inactivating mutations in BAP1 have been previously associated with tumors of the breast and lung (Jensen, et al.(1998) Oncogene 16(9):1097-1112; Wood, et al. (2007) Science 318(5853):1108-1113; Buchhagen, Qiu, & Etkind (1994) Intl J Cancer 57(4):473-479) and, consistent with BAP1's role as tumor suppressor, restoration of BAP1 function has been shown to suppress cell growth and tumorigenicity in a BAP1-mutant lung cancer cell line (Ventii, et al. (2008) Cancer Res 68(17):6953-6963)).

Recently, Harbour et al. reported that inactivating mutations in BAP1 were found at high frequency (26 of 31; 84%) in class 2 (high metastatic risk) uveal melanomas vs. class 1 (low metastatic risk) uveal melanomas (1 of 26; 4%), implicating mutational inactivation of BAP1 as a key event in the acquisition of metastatic competence in uveal melanoma (Harbour et al. (2010) Science 330(6009):1410-1413). The BAP1 mutations associated with uveal melanoma were almost exclusively of somatic origin: Only one patient (with class 2) uveal melanoma exhibited a germline mutation in BAP1.

Although BAP1 has been previously identified as a tumor suppressor and mutations in BAP1 have been previously associated with metastatic competence in uveal melanoma, inactivation of BAP1 has not been previously associated with melanocytic neoplasms of the skin. Further, germline mutations in BAP1 been not been previously associated with susceptibility to cancer or to melanocytic neoplasms.

In view of the above, new markers and methods for use in the accurate diagnosis, prognosis, and/or monitoring of patients with melanocytic nevi or with melanoma are urgently needed. The markers and methods of the present invention address this need.

The citation of references herein shall not be construed as an admission that such is prior art to the present invention.

### SUMMARY OF THE INVENTION

The invention relates generally to the identification of a novel tumor suppressor and marker, a tumor susceptibility gene, in cancers, particularly melanoma. The invention identifies and characterizes a tumor suppressor gene and marker, the BRCA1 -associated protein-1 gene (ubiquitin carboxy-terminal hydrolase; BAP1), which if mutated or altered, particularly to be non-functional or unexpressed, results in increased risk for malignant transformation or cancer, particularly melanoma. The expression or allelic status of BAP1 can be utilized in characterizing, assessing and staging skin lesions, including skin biopsy material and samples. Genetic assessment of the BAP1 gene and/or determination of the expression of the BAP1 protein can be used in assessing, evaluating and characterizing an individual or a lesion, particularly a skin lesion, including for risk of malignancy. The invention now identifies BAP1 as a tumor suppressor gene for skin cancer and melanoma, including melanomas of the skin and uveal melanoma. BAP1 is also associated with mesothelioma, particularly in the absence of exposure to asbestos or erionite.

This invention is drawn to methods and compositions for identifying individuals at risk for melanocytic nevi and/or melanomas, for distinguishing between nevi at high and low risk for malignant transformation, and for the diagnosis, prognosis and treatment of melanocytic neoplasms and/or melanomas.

The invention provides a method for diagnosing or prognosing melanoma in a human comprising determining the expression of BAP1 protein and/or DNA mutation status of the BAP1 gene in said human. In accordance with the method, a diagnosis or prognosis of melanoma is determined wherein BAP1 protein is not expressed and/or wherein the BAP1 gene is mutated so as to alter activity or expression of BAP1. In a particular aspect the expression of BAP1 protein is determined in a biopsy or skin tumor sample. In a particular aspect, the DNA mutation status of the BAP1 gene is determined in one or more of a biopsy or skin tumor sample, particularly for a somatic mutation, and a normal skin sample, cellular sample, or blood sample, particularly for a germline mutation.

The invention includes methods of screening for mutations of the BAP1 gene in a subject or sample for diagnosing or determining predisposition to melanocytic neoplasms and melanoma. Assays and kits for diagnosing or determining predisposition to or risk of melanocytic neoplasms and melanoma in a subject are provided wherein BAP1 protein expression or activity and/or the sequence of the BAP1 gene or existence of BAP1 mutations are determined.

The present invention is directed to novel methods for determining predisposition to or risk of melanoma in a subject by determining the expression or activity of BAP1 protein and/or the genotype or sequence of the BAP1 gene. In an aspect of the method, the germline BAP1 genotype is determined, whereby BAP1 genotype or sequence of normal cells, including in particular normal skin cells, of the subject is determined. In an aspect of the method the somatic BAP1 genotype is determined whereby BAP1 genotype or sequence of skin lesion cells is determined, and wherein the genotype of skin lesion cells differs from the genotype of normal cells, particularly normal non lesion skin cells, indicating a somatic mutation. In accordance with the present invention, BAP1 mutation in skin lesion cells indicates that the lesion is at risk of melanocytic progression. In an aspect of the method the expression or activity of BAP1 protein is determined or assessed using an antibody specific for BAP1, such as in determining binding of labeled antibody or in immunohistochemistry of a skin or biopsy sample.

The invention provides methods for characterizing and classifying a skin lesion comprising determining the expression or activity of BAP1 protein in a sample or biopsy of the skin lesion. In an aspect of the method the expression or activity of BAP1 protein is determined or assessed using an antibody specific for BAP1, such as in determining binding of labeled antibody or in immunohistochemistry of a skin or biopsy sample. In a further aspect of the method a skin lesion is characterized or classified by determining the genotype or sequence of the BAP1 gene. In one such aspect, the germline BAP1 genotype is determined, whereby BAP1 genotype or sequence of normal cells, including in particular normal skin cells, of the subject is determined. In a further aspect of the method the somatic BAP1 genotype is determined whereby BAP1 genotype or sequence of skin lesion cells is determined, and wherein the genotype of skin lesion cells differs from the genotype of normal cells, particularly normal non lesion skin cells, indicating a somatic mutation. In accordance with the present invention, BAP1 mutation in skin lesion cells indicates that the lesion is at risk of melanocytic progression.

Additional tumor suppressor genes or tumor markers may be assessed or determined in accordance with and as an additional aspect of the method, including assessment or determination of BRAF genotype or mutations. Additional tumor suppressor genes or tumor markers for assessment or determination in combination or conjunction with BAP1 may be selected from BRAF, KIT, HRAS, GNAQ and GNA11.

In particular, the invention relates to methods and materials used to isolate and detect BAP1, particularly human BAP1, including primer pairs for DNA, genetic and gene analysis. Numerous exemplary mutant alleles of BAP1 which cause or are associated with susceptibility to benign and malignant melanocytic neoplasms are provided herein. More specifically, the invention provides exemplary and particular germline mutations in the BAP1 gene and their use in the diagnosis of predisposition to melanocytic nevi and/or melanomas. The present invention further provides exemplary and particular somatic mutations in BAP1 in melanocytic nevi and/or melanomas and their use in the diagnosis, classification and prognosis of melanocytic nevi and/or melanomas. Additionally, the invention relates to somatic mutations in the BAP1 gene in other neoplasms and their use in the diagnosis, prognosis and treatment of such neoplasms.

In an aspect of the invention, a method for determining or predicting risk for melanoma or for diagnosing or prognosing melanoma in a subject, particularly a human, is described which comprises:

(a) isolating a sample from the subject, wherein the sample comprises expressed proteins and amplifiable nucleic acid sequences;

(b) contacting the sample with an antibody which specifically binds BAP1 protein and determining whether binding has occurred, wherein antibody binding indicates that active or expressed BAP1 protein is present in the sample and the absence of antibody binding indicated that BAP1 protein is not active or expressed in the sample; and wherein the indication that BAP1 protein is not active or expressed in the sample is a positive indicator of risk or likelihood of melanoma in the subject.

In a further aspect thereof, the method additionally comprises:

(c) performing an amplification reaction of the nucleic acid sequences of the sample, wherein the amplification reaction comprises at least one primer pair capable of annealing to and amplifying BAP1 sequence wherein the amplification reaction is capable of producing a BAP1 specific amplification product; and sequencing the amplification product produced by the amplification reaction to determine the BAP1 sequence, wherein detection of a BAP1 mutant sequence in the amplification product is a positive indicator of risk or likelihood of melanoma in the subject.

In one embodiment, the sample is a skin biopsy or fixed tissue from a subject, such as from a skin lesion. For determining genomic mutations of BAP1, the sample may be normal skin cells, any normal tissue or cells, or may be peripheral blood or peripheral blood cells. Peripheral blood samples may be further processed to remove erythrocytes, thereby producing a population of erythrocyte-depleted peripheral blood cells.

In accordance with the method of the invention, the subject may be a mammal. In a particular aspect, the subject is a human. In a particular aspect, the subject is an adult human. In a particular aspect and with regard to screening for a germline mutation, the subject sample may be a prenatal or embryonic sample.

In an aspect of the method, the BAP1 mutation may be selected from a mutation provided herein, such as in any of the Tables or Figures hereof, including Tables 1-4, or may be any mutation which results in reduced or altered BAP1 activity or expression. Exemplary primers and primer pairs capable of amplifying exon regions or BAP1 for determining the gene sequence are provided herein, for example in Table 5, SEQ ID NOS: 3-40. Alternative primers may determined or designed based on the nucleic acid sequence of BAP1, which is known to those skilled in the art, as provided in FIG. 10 hereof and in SEQ ID NO: 2.

The invention also relates to the treatment, monitoring and therapy of melanomas or melanocytic lesions that have one or more mutation in the BAP1 gene. Thus, a method is provided to treat melanocytic nevi or melanomas compromising restoring, to the neoplastic cells, the function of the protein encoded by the BAP1 gene. In an aspect of the method, BAP1 protein is introduced into the neoplastic cells. In another aspect of the method, DNA encoding BAP1 protein is introduced into the neoplastic cells.

The invention further relates to the screening of drugs for the treatment and/or prevention of melanocytic nevi and/or melanoma, particularly drugs which modulate BAP1 or a BAP1 target. A method to screen for therapeutics to treat melanocytic nevi or melanomas comprising treating neoplastic cells lacking functional BAP1 protein with candidate compounds and selecting those that reverse the neoplastic phenotype of such cells or selecting those that restore a BAP1 protein function.

In the present disclosure, the invention is described with reference to specific embodiments thereof. It will, however, be evident that various modifications and changes may be made thereto without departing from the broader scope and spirit of the invention. The summary, description, materials and methods and drawings are, accordingly, to be regarded in an illustrative rather than restrictive sense.

Numerous references have been made to patents and printed publications throughout this document. Each of the cited references and printed publications is individually incorporated herein by reference in its entirety.

Other objects and advantages will become apparent to those skilled in the art from a review of the following description which proceeds with reference to the following illustrative drawings.

### BRIEF DESCRIPTION OF THE DRAWINGS

FIG. 1A, 1B and 1C. Families with susceptibility to develop multiple melanocytic nevi. Pedigrees, clinical and histopathologic findings for family 1 (left column) and family 2 (right column). In the pedigrees of families 1 and 2, circles represent women, squares men, open symbols unaffected probands, solid symbols clinically affected probands with multiple epithelioid nevi, the “*” indicates probands with uveal melanoma and “* *” probands with cutaneous melanoma (Panel A). Representative nevi from three affected probands of each family. Note the characteristic amelanotic, dome-shaped appearance (Panel B). Histopathology of representative nevi from both families showing relatively symmetrical intradermal proliferations composed of large epithelioid melanocytes with abundant cytoplasm and varying degrees of nuclear pleomorphism (Panel C).

FIG. 2A, 2B and 2C. Pedigree of family 1 and segregation of BAP1 mutations in both pedigrees. The extended pedigree of family 1 is shown in Panel A. The pedigrees of family 1 (Panel B) and family 2 (Panel C) show the segregation of the two BAP1 mutations. Circles represent women, squares represent men, solid symbols clinically affected patients, and open symbols unaffected patients.

FIG. 3-1A to 3-1F and FIG. 3-2A to 3-2F. Histopathological features of representative nevi. Low magnification images of six representative nevi, showing symmetrical intradermal melanocytic proliferations (left column, Panel A, C, E, G, I, K). Corresponding high magnification images in the right column show large epithelioid melanocytes with abundant cytoplasm and varying degrees of nuclear pleomorphism with scattered multinuclear cells (Panel B, D, F, H, J, K).

FIG. 4A to 4C. Genetic findings resulting in the identification of BAP1 mutations. aCGH profiles of melanocytic nevi from family 1. The uppermost profile illustrates the deletion of chromosome 3 in its entirety, whereas the other profiles show focal deletions in 3p. The minimally deleted region (black arrows) encompasses approximately 6 megabases (Panel A). Germline BAP1 mutations in representative probands from both families and corresponding wild type sequences in unaffected members (Panel B). Panel C shows representative examples of inactivation of the second BAP1 allele in three nevi from family 1. Upper left: Homozygous mutation in a tumor with 3p21 loss. Upper right: Homozygous mutation in a tumor without 3p21 loss, suggesting presence of maternal uniparental disomy. Lower left and right: Compound heterozygosity through mutation of the BAP1 wild type allele.

FIG. 5A and 5B. Linkage analysis and loss of paternal SNPs in tumors showing a loss of chromosome 3. Pedigree of family 1 showing generations I and II as in FIG. 1A. The haplotypes of the father (I-1) are shown in gray and those of the mother (I-2) in blue. The corresponding haplotypes of the children were reconstructed based on 250K Affymetrix SNP microarray results. Note that the three affected children (II-2, II-4, and II-8) all have the same maternal allele for the candidate region (dark blue), whereas the non-affected son (II-6) inherited the other maternal allele (light blue). Parametric linkage analysis of these six probands yielded a positive LOD score of 0.9 (Panel A). SNP arrays of tumors with chromosome 3 loss confirmed that the paternal copy carrying wild-type allele of BAP1 was lost, while the maternal copy harboring the mutated allele was retained (Panel B).

FIG. 6. BAP1 mutation (c.1305delG) in family 1. Illustration of massively parallel sequencing performed by a SOLiD 4 device using the Integrative Genomics Viewer (http://www.broadinstitute.org/igv). Proband 1-2 harbors a 1 base pair deletion in exon 13 of BAP1, which is evident in the sequence reads (gray arrows). The unaffected proband I-1 does not exhibit this deletion.

FIG. 7A, 7B and 7C. Consequences of BAP1 mutations on mRNA splicing. Control RNA and RNA from two probands from family II (II-6, III-3) was reverse transcribed into cDNA, amplified with PCR primers annealing to exons 13 and 17, and separated on 1% agarose gel. While reverse transcription and PCR amplification of the control RNA results in a single product of the predicted size (355 bp) containing only the exons (introns are spliced out), reverse transcription and PCR amplification of RNA from the two affected probands results in two products: One is the correctly spliced form (355 bp); the other, a consequence of the splice site mutation, is an abnormal PCR product of 535 by (355+180 by of intron 16) (Panel A). Anti-BAP1 antibody stains nuclei in epidermal keratinocytes (internal positive control), but not in the melanocytic tumor cells, indicating a loss of BAP1 expression in both tumor from family 1 (Panel B) and tumor from family 2 (Panel C).

FIG. 8A to 8C. Progression of a nevus (N) to melanoma (M) is associated with a loss of BAP1. Scanning magnification of a melanoma arising within a nevus (Panel A). Nevus component with bland melanocytes with monomorphous round to ovoid nuclei (Panel B). Melanoma component showing melanocytes with large pleomorphic nuclei and prominent nucleoli (Panel C). MIB-1 immunohistochemistry at the interface of nevus and melanoma areas showing low to absent labeling in the nevus compared to the melanoma (Panel D). Immunohistochemistry for BAP1 at the interface of nevus and melanoma with conspicuous nuclear staining in the nevus contrasted with absent nuclear staining in the melanoma (Panel E). The nevus component shows a BRAF^{V600E} mutation, but no chromosomal aberrations or BAP1 mutation (Panel F). The melanoma component shows focal loss of chromosome region 3p21 spanning the BAP1 locus, a frameshift mutation of the second BAP1 allele, and the identical BRAF mutation seen in the nevus component (Panel G).

FIG. 9 depicts the BAP1 protein amino acid sequence (corresponding to Genbank NP_{—}004676.1) (SEQ ID NO:1).

FIG. 10 depicts the BAP1 nucleic acid sequence (cDNA) (corresponding to Genbank NM_{—}004656.2) (SEQ ID NO:2) and lists the locations of Exons 1-17.

FIG. 11A to 11I Skin tumor from 49-year-old male harboring a germline mutation in BAP1. (A) Dome-shaped to pedunculated, skin-colored tumor. (B) Polypoid, relatively symmetrical, dermal tumor consisting of (C) melanocytes arranged in nests and sheets. (D) The neoplastic cells are characterized by moderate amounts of amphophilic cytoplasm, well-defined cytoplasmic borders, and pleomorphic oval nucleoli. (E) BAP1 IHC is negative in tumor cells, but positive in epidermal keratinocyte nuclei and in scattered lymphocytes within the tumor. (F) Fluorescence in-situ hybridization shows loss of the second BAP1 allele in tumor cell nuclei: one red signal (chr. 3p21, BAP1 locus), one orange signal (chr. 3p25), and 2 green control signals per nucleus. (G) Array CGH shows a loss of the whole chromosome 3 with no other chromosomal aberrations. (H) The sequencing electropherograms show a BRAF^{V600E} mutation and (I) the germline BAP1 mutation. The wild-type BAP1 sequence is markedly reduced indicating a loss of the wild-type allele in the tumor.

FIG. 12A through 12F. Atypical Spitz tumor from the shoulder of a 23-year-old male. (A) Polypoid, predominantly dermal melanocytic tumor composed of (B) large epithelioid cells with numerous admixed lymphocytes. (C) The tumor cells contain abundant amphophilic cytoplasm with well-defined cytoplasmic borders and pleomorphic, round to oval, vesicular nuclei with conspicuous nucleoli. Tumor-infiltrating lymphocytes are prominent. (D) The epithelioid cells do not express BAP1, while the admixed lymphocytes are strongly positive for BAP1. (E) Sequencing electropherograms show a BRAF^{V600E} mutation and (F) an inactivating, frameshift BAP1 mutation (c.459del, p.E154Rfs*33) in the tumor.

FIG. 13A, 13B and 13C. Atypical Spitz tumor from the back of 19-year-old patient. (A) Shave biopsy of a relatively symmetric dermal tumor composed of (B) sheets and nests of large epithelioid cells. Inset: No BAP1 staining in the nuclei of the epithelioid cells, while the admixed lymphocytes are strikingly positive. (C) The tumor cells contain abundant amphophilic cytoplasm with well-defined cytoplasmic borders and very pleomorphic round/oval vesicular nuclei with conspicuous nucleoli. Occasional multinucleate tumor cells are present. Note the prominent tumor-infiltrating lymphocytes.

FIG. 14A through 14E. Atypical Spitz tumor from the upper back of 51-year-old female. (A) Symmetrical dermal tumor composed of (B) sheets of large epithelioid cells. The tumor cells contain abundant amphophilic cytoplasm with well-defined cytoplasmic borders and pleomorphic vesicular nuclei with conspicuous nucleoli. Occasional multinucleate tumor cells are present. Only few tumor-infiltrating lymphocytes are present. Inset: Absent nuclear staining for BAP1 in the epithelioid cells, with some positive admixed lymphocytes. (C) Array CGH of the tumor shows a focal loss of the BAP1 region on chromosome 3 and a loss of the long arm of chromosome 16. (D) The sequencing electropherograms show a BRAF^{V600E} mutation and (E) an inactivating frameshift mutation of BAP1 (BAP1: c.920dup, p.N308Qfs*90).

FIG. 15A through 15F. Atypical Spitz tumor from a 59-year-old male. (A) Symmetrical, melanocytic tumor (B) with no epidermal involvement. (C) Medium to large epithelioid cells with abundant amphophilic cytoplasm and pleomorphic, vesicular nuclei with conspicuous nucleoli. Insert: Absent nuclear BAP1 staining in large epithelioid melanocytes with strong nuclear staining in admixed lymphocytes. (D) Array CGH shows a loss of the entire chromosome 3. (E) Sequencing revealed no BRAF^{V600E} mutation (wild type), but (F) a missense BAP1 mutation (c.516C>G, p.S172R) in the tumor.

FIG. 16A through 16D. Atypical Spitz tumor from the upper back of a 44-year-old female. (A) Small polypoid melanocytic tumor (B) with medium to large epithelioid cells and admixed lymphocytes. Inset: Absent BAP1 staining in the melanocytes, but strong nuclear staining in admixed lymphocytes. (C) Sequencing electropherograms show a BRAF^{V600E} mutation and an inactivating, nonsense mutation of BAP1 (c.178C>T, p.R60*). (D) Array CGH shows a balanced profile with no gains and losses.

FIG. 17A, 17B and 17C. Atypical Spitz tumor from the arm of a 37-year-old patient. (A) Dome-shaped melanocytic tumor (B) composed of sheets of medium sized, spindle-shaped, oval and epithelioid melanocytes with amphophilic cytoplasm, but lacking distinct cytoplasmic borders. (C) Strong expression of BAP1 in the keratinocytes and in the melanocytes. This tumor showed a _{BRAF}^{V600}E mutation (data not shown).

FIG. 18A through 18D. (A) Pedigree of a family in which mesotheliomas were inherited in an autosomal-dominant pattern; (B) melanocytic tumor of subject III-2; (C) hematoxylin-eosin (HE) stain of malignant mesothelioma of subject III-1; (D) hematoxylin-eosin (HE) stain of malignant mesothelioma of subject III-2; BAP1 immunohostochemistry in the mesothelioma from subject II-2 showing loss of nuclear BAP1 staining

FIG. 19A, 19B and 19C. (A) Residual epithelioid nevus intimately associated with base of cutaneous melanoma subsequently diagnosed in subject II-1 of Family I in Example 1; (B) The hematoxylin-eosin (HE) stain shows the nevus component with epitheloid cells and moderate nuclear polymorphism, and (C) the melanoma component with large pleiomorphic melanocytes, vesicular nuclei, prominent nucleoli and mitotic figures (arrows).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

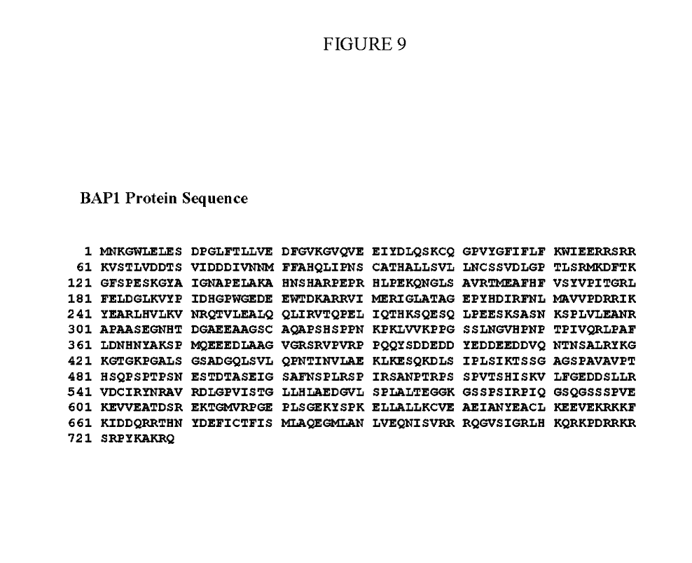

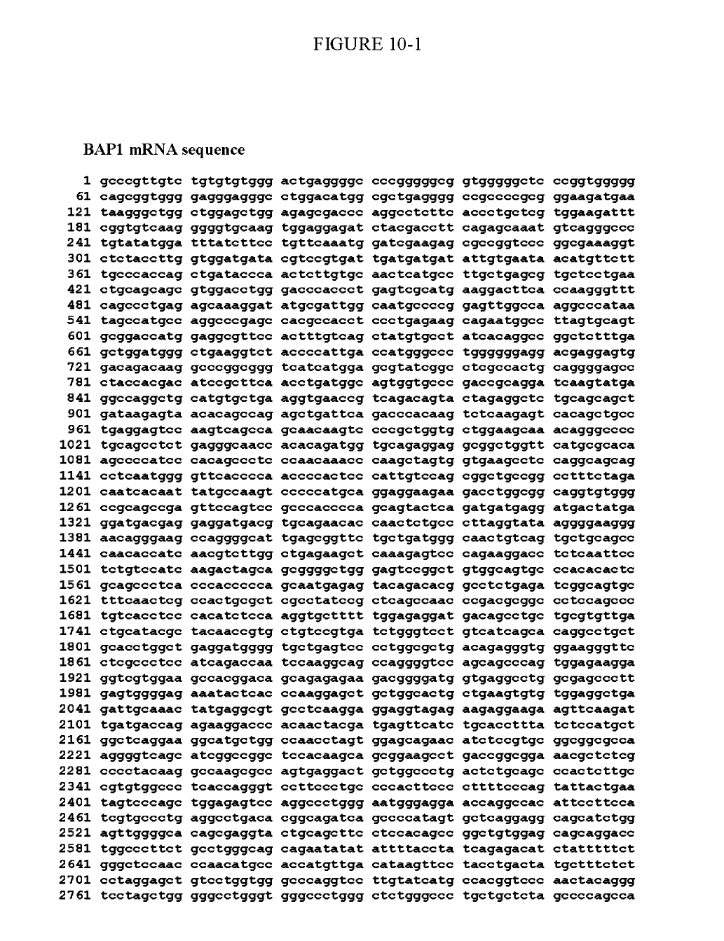

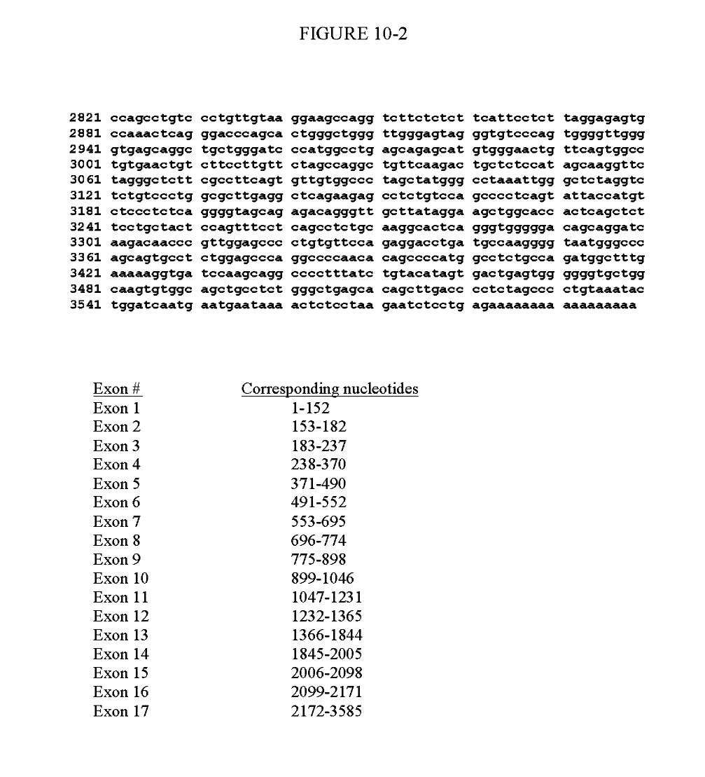

The terms “BRCA1-as so ciated protein-1”, “BRCA1-as so ciated protein-1 gene”, “ubiquitin carboxy-terminal hydrolase”, “BAP1”, “BAP1” and any variants not specifically listed, as used herein and throughout the present application and claims refer a tumor susceptibility or tumor suppressor protein and encoding gene, including proteinaceous material and nucleic acid material, having the characteristics and profile of activities, uses or applications set forth herein and in the Claims. The term “BAP1” refers particularly to proteinaceous material and extends to those proteins having the amino acid sequence data described and referred to herein and provided in FIG. 9 (SEQ ID NO:1), and the profile of activities, uses or applications set forth herein and in the Claims. The term “BAP1” refers to nucleic acid material, particularly BAP1 encoding nucleic acids, and extends to those nucleic acids having the sequence data described herein and provided in FIG. 10 (SEQ ID NO:2), and the profile of activities, uses or applications set forth herein and in the Claims. Accordingly, proteins or nucleic acids displaying substantially equivalent or altered activity are likewise contemplated. In particular, mutations and mutants of BAP1 protein and BAP1 encoding nucleic acids are described and provided herein, particularly including BAP1 mutations which result in altered activity or expression of BAP1. These modifications may be deliberate, for example, such as modifications obtained through site-directed mutagenesis, or may be accidental, inherited (such as genomic mutations) or acquired (such as somatic mutations). The terms “BRCA1-associated protein-1”, “BRCA1-associated protein-1 gene”, “ubiquitin carboxy-terminal hydrolase”, “BAP1”, “BAP1” are intended to include within their scope proteins and nucleic acids specifically recited herein as well as all substantially homologous analogs, mutants or mutations, and allelic variations.

The term “melanoma” as used herein may be used to refer to either primary melanoma or metastatic melanoma, and includes all clinically recognized and accepted forms of melanoma, including cutaneous melanoma and uveal melanoma.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the -10 and -35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

The term “substantially pure” refers to a preparation comprising at least 50-60% by weight of the compound of interest (e.g., nucleic acid, oligonucleotide, protein, etc.). More preferably, the preparation comprises at least 75% by weight, and most preferably 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide or amino acid means a sequence having the properties of a given SEQ ID NO:. For example, when used in reference to an amino or nucleic acid sequence, the phrase includes the sequence per se and molecular modifications that would not affect the basic and novel characteristics of the sequence. With regard to the novel primers of the present invention, for example, the phrase includes the sequence per se and molecular modifications that would not appreciably affect the ability of the primer to act as a BAP1 specific primer.

The term “percent identical” is used herein with reference to comparisons among nucleic acid or amino acid sequences. Nucleic acid and amino acid sequences are often compared using computer programs that align sequences of nucleic or amino acids thus defining the differences between the two. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (at http://www.ncbi.nlm.nih.gov/blast/; Altschul et al., 1990, J Mol Biol 215:403-410) using a gapped alignment with default parameters, may be used to determine the level of identity and homology between nucleic acid sequences and amino acid sequences.

With respect to single stranded nucleic acids, particularly oligonucleotides, the terms “specifically hybridizing” or “specifically annealing” refer to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

The term “oligonucleotide,” as used herein refers to primers and probes of the present invention and of use in the present invention, and is defined as a nucleic acid molecule comprised of two or more ribo- or deoxyribonucleotides, preferably more than three. The exact size of the oligonucleotide will depend on various factors and on the particular application and use of the oligonucleotide. Preferred oligonucleotides comprise 15-50 consecutive bases. Exemplary BAP1-specific PCR primers of the present invention are nucleic acid sequences comprising one or more of SEQ ID NOs:3-40. The present invention also encompasses BAP/-specific PCR primers which share at least about 80% homology with nucleic acids of any one of SEQ ID NO:3-40.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “functional” as used herein implies that the nucleic or amino acid sequence is functional for the recited assay or purpose.

The term “biologically compatible salt solution” is used herein to describe any salt solution in which nucleic acid sequences and/or proteins may be stably maintained. For applications wherein nucleic acid sequences in such salt solutions are to be amplified, such salt solutions are modifiable (e.g., capable of being diluted or altered to change substituent concentration) to be compatible with polymerase activity and the like.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding a protein of the invention, such as BAP1, which code for a having the same amino acid sequence as SEQ ID NO:1 for example, but which are degenerate to SEQ ID NO:2. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab', F(ab')₂ and F(v), which portions are preferred for use in the therapeutic methods described herein. Fab and F(ab')₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab' antibody molecule portions are also well-known and are produced from F(ab')₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

Methods for producing polyclonal anti-polypeptide antibodies are well-known in the art. See U.S. Pat. No. 4,493,795 to Nestor et al. The general methodology for making monoclonal antibodies by hybridomas is well known. A monoclonal antibody, typically containing Fab and/or F(ab')₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

As used herein, “pg” means picogram, “ng” means nanogram, “ug” or “μg” mean microgram, “mg” means milligram, “ul” or “μl” mean microliter, “ml” means milliliter, “1” means liter.

Despite recent advances in the study of melanoma biology, the development of molecular tools useful for diagnosing and/or monitoring patients with melanoma is still developing. Few advances have been made in protocols designed to monitor patients for primary disease or disease recurrence, or to select patients at high risk for the development of melanoma or melanocytic lesions. The absence of improved prognostic tools for such assessments makes it difficult for attending physicians to determine the best treatment strategies or to identify and readily monitor those most at risk of disease.

In accordance with the invention, mutational events in the BAP1 gene are now recognized as being particularly associated with melanoma and can be utilized in diagnosis or prognosis of skin lesions, including melanoma. The mutations of the BAP1 gene can involve gross rearrangements, such as insertions and deletions, or may be point mutations which alter the sequence of the expressed protein or result in premature termination or alternative splicing such that no protein or a shortened or inactive protein is produced.

In its primary aspect, the present invention concerns the identification of a novel tumor suppressor and marker, a tumor susceptibility gene, the BRCA1-associated protein-1 gene (ubiquitin carboxy-terminal hydrolase; BAP1), in melanoma and other cancers, which if mutated or altered, particularly to be non-functional or unexpressed, results in increased risk for malignant transformation or cancer, particularly melanoma, including melanomas of the skin and eye. Genetic assessment of the tumor suppressor gene and/or determination of the expression of the protein can be used in assessing, evaluating and characterizing an individual or a lesion, particularly a skin lesion, including for risk of malignancy. Mutations of BAP1 gene and/or altered or no expression of BAP1 protein are associated with risk of melanoma. Mutations of BAP1 gene and/or altered or no expression of BAP1 protein are also associated with risk of mesothelioma, particularly in the absence of exposure to asbestos or erionite.

This invention is drawn to methods and compositions for identifying individuals at risk for melanocytic nevi and/or melanomas, for distinguishing between nevi at high and low risk for malignant transformation and for the diagnosis, prognosis and treatment of melanocytic neoplasms and/or melanomas. The invention provides a method for diagnosing or prognosing melanoma in a human comprising determining the expression of BAP 1 protein and/or DNA mutation status of the BAP1 gene in said human. In accordance with the method, a diagnosis or prognosis of melanoma is determined wherein BAP 1 protein is not expressed and/or wherein the BAP 1 gene is mutated so as to alter activity or expression of BAP1.

According to the diagnostic and prognostic method of the present invention, alteration of the wild-type BAP1 gene is detected and/or the absence of BAP1 protein is determined. “Alteration of a wild-type gene” according to the present invention encompasses all forms of mutations-including deletions, substitutions, terminations, splice alterations. The alteration may be due to rearrangements such as insertions, inversions, and deletions, or to point mutations. Deletions may be of the entire gene or only a portion of the gene. Somatic mutations are those which occur only in certain tissues, e.g., in the tumor tissue, and are not inherited in the germline. Germline mutations can be found in any of a body's tissues. If only a single allele is somatically mutated, an early neoplastic state is indicated. However, if both alleles are mutated then a late neoplastic state is indicated. The finding of BAP1 mutations thus provides both diagnostic and prognostic information. A BAP1 allele which is not deleted (e.g., that on the sister chromosome to a chromosome carrying an BAP1 deletion) can be screened for other mutations, such as insertions, small deletions, and point mutations. It is believed that many mutations found in tumor tissues will be those leading to decreased expression of the BAP1 gene product, as indicated and demonstrated herein. However, mutations leading to non-functional gene products may also lead to a cancerous or pre-cancerous state. Point mutational events may occur in regulatory regions, such as in the promoter of the gene, leading to loss or diminution of expression of the mRNA and loss or no significant or detectable BAP1 protein product. Point mutations may also abolish proper RNA processing, leading to loss of expression of the BAP1 gene product.

BAP 1 protein and/or BAP1 nucleic acid is assessed using methods of the art. The expression of BAP1 protein is determined in a biopsy or skin tumor sample. The DNA mutation status of the BAP1 gene is determined in one or more of a biopsy or skin tumor sample, particularly for a somatic mutation, and a normal skin sample, cellular sample or blood sample, particularly for a germline mutation. In order to detect the alteration of the BAP1 gene in a tissue or lesion, the lesion tissue is isolated free from surrounding normal tissues. Means for enriching a tissue preparation for tumor cells are known in the art. For example, the tissue may be isolated from paraffin or cryostat sections. Cancer cells may also be separated from normal cells by flow cytometry. These as well as other techniques for separating tumor cells from normal cells are well known in the art.

DNA may be isolated from clinical specimens, particularly tissue and biopsy specimens, using methods known and described or accepted in the art. For example, herein a modified version of the Qiagen QIAamp DNA FFPE Tissue Kit (Qiagen, Hilden, Germany) or the chemagic DNA Tissue Kit (Chemagen, Baesweiler, Germany) is utilized according to the manufacturers' instructions for DNA isolation. When working with isolated cells from a subject or from a blood sample, standard DNA isolation procedures may be utilized, including those which are designed for cell and not for tissue samples, such as the Qiagen QIAmp DNA Mini Kit.

Alterations in BAP1 sequence may be detected using genomic hybridization techniques, such as array-based comparative genomic hybridization, as exemplified and utilized herein, wherein a test or subject sample and a reference sample of DNA are differentially labeled, for example with different colored dyes, and sequence changes, differences or gaps grossly identified by changes in dye or color. Detection of point mutations may be accomplished by molecular cloning of the BAP1 allele (or alleles) and sequencing that allele(s) using techniques well known in the art. Alternatively, amplification, such as via the polymerase chain reaction (PCR), can be used to amplify gene sequences directly from a genomic DNA preparation from the tumor tissue. The DNA sequence of the amplified sequences can then be determined. The polymerase chain reaction itself is well known in the art. See, e.g., Saiki et al., Science, Vol. 239, p. 487, 1988; U.S. Pat. Nos. 4,683,203, 4,683,195, 4,800,195, and 4,965,188, the entire disclosures of which are incorporated by reference herein. Specific primers which can be used in order to amplify the gene can be generated or designed and exemplary primers are provided herein. The ligase chain reaction, which is known in the art, can also be used to amplify APC sequences. See Wu et al., Genomics, Vol. 4, pp. 560-569 (1989). In addition, a technique known as allele specific PCR can be applied (see Ruano and Kidd, Nucleic Acids Research, Vol. 17, p. 8392, 1989) wherein primers are used which hybridize at their 3′ ends to a particular BAP1 mutation. If the particular BAP1 mutation is not present, an amplification product is not observed. Amplification Refractory Mutation System (ARMS) can also be used as disclosed in European Patent Application Publication No. 0332435 and in Newton et al., Nucleic Acids Research, Vol. 17, p.7, 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening among kindred persons of an affected individual for the presence of the BAP1 mutation found in that individual. Single stranded conformation polymorphism (SSCP) analysis can also be used to detect base change variants of an allele. (Orita et al., Proc. Natl. Acad. Sci. USA Vol. 86, pp. 2766-2770, 1989, and Genomics, Vol. 5, pp. 874-879, 1989.) Other techniques for detecting insertions and deletions as are known in the art can be used. Point mutations may be detected by amplifying and sequencing the mRNA or via molecular cloning of cDNA made from the mRNA. The sequence of the cloned cDNA can be determined using DNA sequencing techniques which are well known in the art. The cDNA can also be sequenced via the polymerase chain reaction (PCR) as known in the art and described herein.

Alteration of wild-type BAP1 genes can also be detected by screening for alteration of wild-type BAP1 protein. Assessment of BAP1 protein may be accomplished for example using immunohistochemistry of tissue or biopsy samples. For example, monoclonal antibodies immunoreactive with BAP1 can be used to screen a tissue or cellular sample. Lack of cognate antigen would indicate a BAP1 or BAP1 mutation. Antibodies specific for products of mutant alleles could also be used to detect mutant BAP1 gene product. Such immunological assays can be done in any convenient format known in the art. These include Western blots, immunohistochemical assays and ELISA assays. Any means for detecting an altered BAP1 protein can be used to detect alteration of wild-type BAP1 gene(s). Functional assays can be used, such as protein binding determinations. In addition, assays can be used which detect BAP1 biochemical function, biological function, activity or signaling.

Preferably, the anti-BAP1 antibody used in the diagnostic methods of this invention is an affinity purified polyclonal antibody or a monoclonal antibody and suitable for assessment of proteins in tissue or tumor samples. More preferably, the antibody is a monoclonal antibody (mAb). The antibody may be labeled or provided with a secondary label suitable for assay, such as suitable for immunohistochemistry. The anti-BAP1 antibody molecules used herein may be in the form of Fab, Fab', F(ab')₂ or F(v) portions of whole antibody molecules. Antibodies recognizing or specific for human BAP1 are particularly preferred. Exemplary BAP1 antibody used herein is clone C-4 available from Santa Cruz Biotechnology. BAP1 antibodies, particularly for immunohistochemistry, may be obtained from other commercial antibody sources, such as ABCAM (Cambridge, Mass.), AB Nova (Taiwan). One of skill in the art can readily make and/or test a BAP1 antibody for suitability and use in assessing BAP1 protein, particularly in skin lesions or biopsy samples.

The presence of BAP1 in cells can be ascertained by the usual immunological procedures applicable to such determinations. A number of useful procedures are known. Three such procedures which are especially useful utilize either BAP1 antibody labeled with a detectable label, or a secondary antibody or binding agent labeled with a detectable label. The procedures and their application are all familiar to those skilled in the art and accordingly may be utilized within the scope of the present invention. Procedures include competitive assays, sandwich assays, double antibody or DASP procedures.

The labels commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The BAP1 antibody or its binding partner(s) can also be labeled with a radioactive element or with an enzyme. The radioactive label can be detected by any of the currently available counting procedures. The preferred isotope may be selected from 3H, 14C₅ 32_{P5} 35s₅ 36_{C15} 51-₁.₅ U ⁵⁷Co, ⁵⁸Co, ⁵⁹Fe, 90_{Y}, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, B-glucuronidase, B-D-glucosidase, B-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

Assays and kits for diagnosing or determining predisposition to or risk of melanocytic neoplasms or melanoma in a subject are provided wherein BAP1 protein expression or activity and/or the sequence of the BAP1 gene or existence of BAP1 mutations are determined. The present invention is directed to novel methods and assays for determining predisposition to or risk of melanoma in a subject by determining the expression or activity of BAP1 protein and/or the genotype or sequence of the BAP1 gene. In an aspect of the method or assay, the germline BAP1 genotype is determined, whereby BAP1 genotype or sequence of normal cells, including in particular normal skin cells, of the subject is determined. In an aspect of the method the somatic BAP1 genotype is determined whereby BAP1 genotype or sequence of skin lesion cells is determined, and wherein the genotype of skin lesion cells differs from the genotype of normal cells, particularly normal non lesion skin cells, indicating a somatic mutation. In accordance with the present invention, BAP1 mutation in skin lesion cells indicates that the lesion is at risk of melanocytic progression. In an aspect of the method the expression or activity of BAP1 protein is determined or assessed using an antibody specific for BAP1, such as in determining binding of labeled antibody or in immunohistochemistry of a skin or biopsy sample.

The invention provides methods for characterizing and classifying a skin lesion comprising determining the expression or activity of BAP1 protein in a sample or biopsy of the skin lesion. In an aspect of the method the expression or activity of BAP1 protein is determined or assessed using an antibody specific for BAP1, such as in determining binding of labeled antibody or in immunohistochemistry of a skin or biopsy sample. In a further aspect of the method a skin lesion is characterized or classified by determining the genotype or sequence of the BAP1 gene. In one such aspect, the germline BAP1 genotype is determined, whereby BAP1 genotype or sequence of normal cells, including in particular normal skin cells, of the subject is determined. In a further aspect of the method the somatic BAP1 genotype is determined whereby BAP1 genotype or sequence of skin lesion cells is determined, and wherein the genotype of skin lesion cells differs from the genotype of normal cells, particularly normal non lesion skin cells, indicating a somatic mutation. In accordance with the present invention, BAP1 mutation in skin lesion cells indicates that the lesion is at risk of melanocytic progression.

In accordance with the invention and methods, additional tumor suppressor genes or tumor markers may be assessed or determined, including assessment or determination of genotype or mutations in one or more genes selected from BRAF, KIT, HRAS, GNAQ and GNAT11. These additional tumor suppressor genes or tumor markers may be assessed or determined in combination or conjunction with BAP1. In a preferred aspect, BRAF is assessed in conjunction or combination with BAP1.

Methods and materials used to isolate and detect BAP1, particularly human BAP1, are provided herein, including primer pairs for DNA, genetic and gene analysis. Numerous exemplary mutant alleles of BAP1 which cause or are associated with susceptibility to benign and malignant melanocytic neoplasms are provided herein. The BAP1 mutation may be selected from a mutation provided herein, such as in any of the Tables or Figures hereof, including Tables 1-4, or may be any mutation which results in reduced or altered BAP1 activity or expression. The invention provides exemplary and particular germline mutations and somatic mutations in the BAP1 gene and their use in the diagnosis of predisposition to melanocytic nevi and/or melanomas. Exemplary primers and primer pairs capable of amplifying exon regions or BAP1 for determining the gene sequence are provided herein, for example in Table 5, SEQ ID NOS: 3-40. Alternative primers may determined or designed based on the nucleic acid sequence of BAP1, which is known to those skilled in the art, as provided in FIG. 10 hereof and in SEQ ID NO: 2. Also contemplated and encompassed by the present invention are novel BAP1 mutant or mutation specific primers, which specifically anneal with and amplify a BAP1 mutation, including any one or more of the mutations provided herein. The mutation specific primer(s) may be used to selectively and specifically amplify nucleic acid in a sample when such mutation is present, thereby providing positive diagnostic indication of the mutation without the need to sequence the BAP1 gene or multiple exonic portions thereof. This approach is feasible upon the determination and characterization of a number of specific and more common allelic mutations associated with the human population.

In an aspect of the invention, a method for determining or predicting risk for melanoma or for diagnosing or prognosing melanoma in a subject, particularly a human, is described which comprises:

(a) isolating a sample from the subject, wherein the sample comprises expressed proteins and amplifiable nucleic acid sequences;

(b) contacting the sample with an antibody which specifically binds BAP1 protein and determining whether binding has occurred, wherein antibody binding indicates that active or expressed BAP1 protein is present in the sample and the absence of antibody binding indicated that BAP1 protein is not active or expressed in the sample; and wherein the indication that Bap1 protein is not active or expressed in the sample is a positive indicator of risk or likelihood of melanoma in the subject.

In a further aspect thereof, the method additionally comprises:

(c) performing an amplification reaction of the nucleic acid sequences of the sample, wherein the amplification reaction comprises at least one primer pair capable of annealing to and amplifying BAP1 sequence wherein the amplification reaction is capable of producing a BAP1 specific amplification product; and sequencing the amplification product produced by the amplification reaction to determine the BAP1 sequence, wherein detection of a BAP1 mutant sequence in the amplification product is a positive indicator of risk or likelihood of melanoma in the subject.

The invention also provides kits for diagnosing, prognosing or evaluating melanoma in a mammal. An exemplary kit contains a container or a sample vial for storing a sample of a tissue or a body fluid; a composition comprising at least one BAP1-specific PCR primer pair in an amount effective to permit detection of mutant BAP1 nucleic acid sequence in a sample; and an instructional material which directs use of the composition for detecting the presence and/or determining the BAP1 mutant nucleic acid sequence. Included in the invention are commercial test kits suitable for use by a medical specialist to determine the presence or absence of BAP1 protein and/or one or more BAP1 gene mutation, so as to diagnose or prognose melanoma or other cancers or melanocytic nevi.

The present invention thus includes a kit for detecting, diagnosing or prognosing melanoma in a mammal which comprises:

a) a container for storing a biological sample obtained from the mammal;

b) a composition comprising at least one BAP1 specific primer pair in an amount effective to permit detection of BAP1 mutant nucleic acid sequence, if present, in said sample and a biologically compatible salt solution;

c) positive and negative control nucleic acid sequences of the BAP/sequence; and

d) an instructional material setting forth a protocol suitable for use in detecting and/or quantifying BAP1 mutant nucleic acid sequences.

As used herein, an “instructional material” includes a publication, recording, diagram, or any other medium of expression that directs or dictates the use of the components of a kit for performing the function of a method of the invention described herein. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

With regard to a composition for inclusion in a kit, detection reagents such as BAP1 specific antibody and BAP1 specific primers and PCR primer pairs are as described herein and may be included in and of use in the inventive methods hereof The composition may therefore comprise at least one set of BAP1 specific PCR primer pairs in an amount effective to permit detection of BAP1 mutant nucleic acid sequence in the sample. Determination of BAP1 sequence and/or detection of BAP1 mutant nucleic acid sequence is accomplished using any of the methods described herein or known to a skilled artisan for determining or detecting a specific nucleic acid molecule in a biological sample or isolated from a biological sample. Exemplary primers are provided herein, including in Table 5 and SEQ ID NOS: 3-40.

In addition to BAP1 specific primer pairs as described above, a kit may also comprise buffers, nucleotide bases, and other compositions to be used in antibody assays, and in hybridization and/or amplification reactions. Each solution or composition may be contained in a vial or bottle and all vials held in close confinement in a box for commercial sale.

The invention further relates to the screening of drugs for the treatment and prevention of melanocytic nevi and/or melanoma, particularly drugs which modulate BAP1 or a BAP1 target. Cells and animals which carry one or more mutant BAP1 allele(s) can be used as model systems to study and test for substances which have potential as therapeutic agents. The cells are typically cultured skin, dermis or epithelial cells. These may be isolated from individuals with BAP1 mutations, either somatic or germline. Alternatively, the cell line can be engineered to carry the mutation in one or more APC allele. After a test substance is applied to the cells, the neoplastically transformed or melanocytic phenotype of the cell will be determined. Any trait of neoplastically transformed or melanoma cells can be assessed, including growth characteristics, pigmentation, anchorage-independent growth, tumorigenicity in nude mice, invasiveness of cells, and growth factor dependence. Assays for each of these traits are known in the art. Animals for testing therapeutic agents can be selected after mutagenesis of whole animals or after treatment of germline cells or zygotes. Such treatments include insertion of one or more mutant BAP1 alleles, as well as insertion of disrupted homologous genes. After test substances have been administered to the animals, the growth of tumors or cellular characteristics are assessed.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Germline and Somatic BAP1 Mutations in Melanocytic Tumors

Hereditary cancer syndromes often stem from germline mutations that result in functional loss of one allele of a tumor suppressor gene. Subsequent somatic cell mutations that result in loss of function of the second allele may then lead to tumor formation, in line with the ‘two-hit hypothesis’ (Knudson, 1971). Well-documented examples of tissue-specific tumor susceptibility resulting from germline mutations in tumor suppressor genes are familial breast cancer (resulting from mutations in BRCA1), familial adenomatous polyposis (resulting from mutations in the adenomatous polyposis coli gene; APC), and retinoblastoma (resulting from mutations in the retinoblastoma 1 gene; RB1) (Garber & Offit, 2005).

Familial cutaneous melanoma is one example of a hereditary skin tumor syndrome. Approximately 10% of all melanoma cases are familial. Approximately one-third of familial cutaneous melanomas are caused by known germline mutations, primarily in cyclin-dependent kinase inhibitor 2A (CDKN2A) and, less frequently, in cyclin-dependent kinase 4 (CDK4) (de Snoo & Hayward, 2005). However, the underlying genetic alterations predisposing to the majority of familial melanocytic tumors are unknown. A combination of genomic analyses, including massively parallel (‘next generation’) sequencing, was used to identify BAP1 as a novel, highly-penetrant gene that predisposes to benign and malignant melanocytic proliferations with unique characteristics.

Clinical and Histopathological Description of Two Families with Multiple Exophytic Melanocytic Nevi

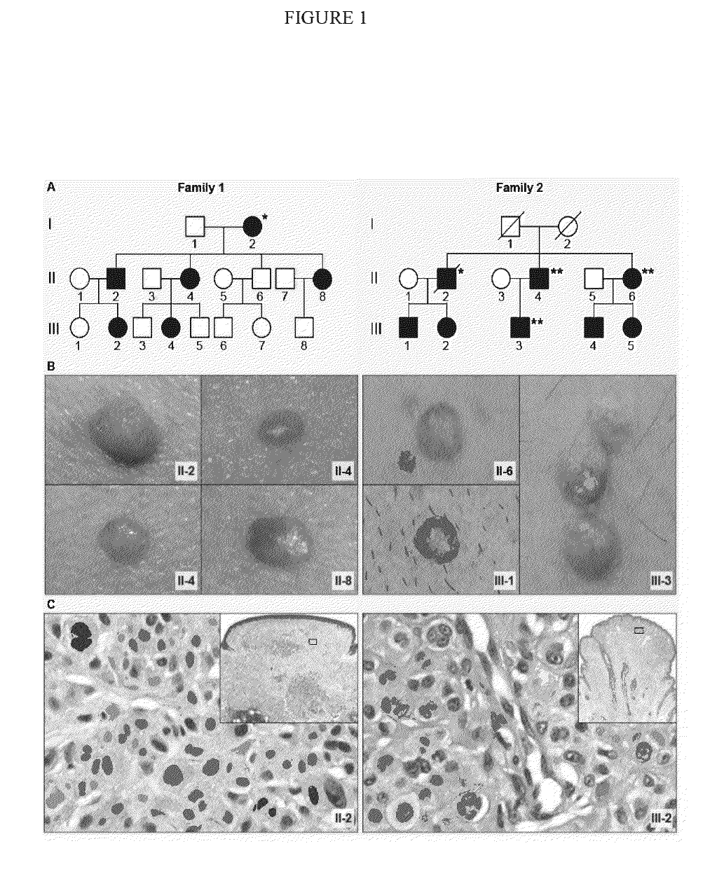

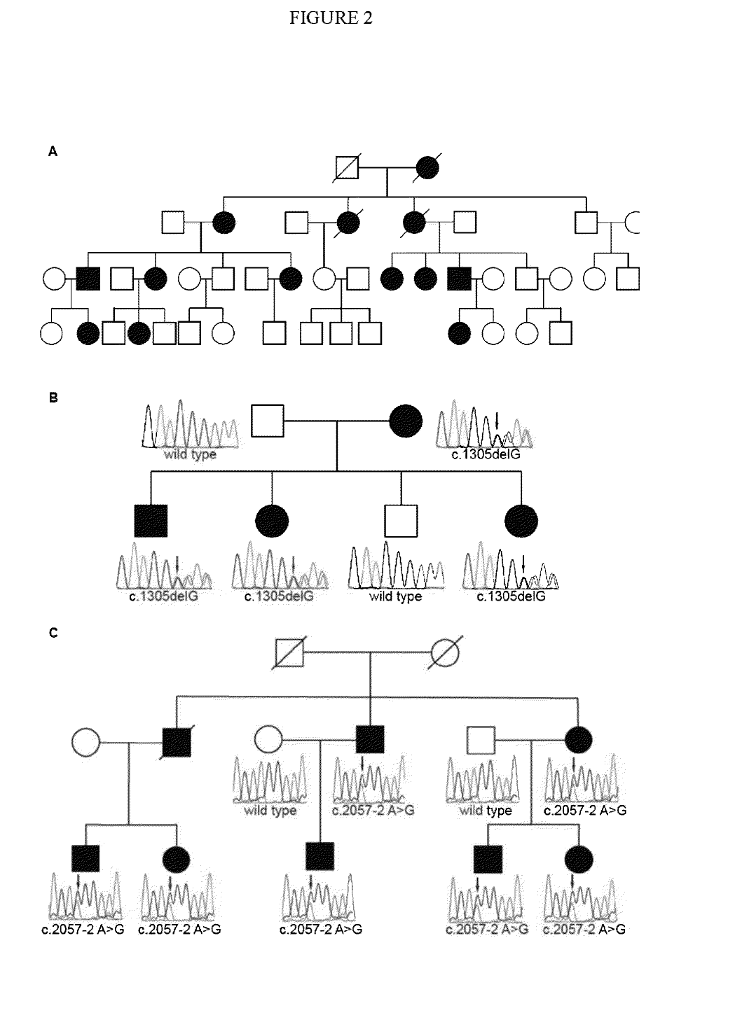

Two families with multiple exophytic melanocytic nevi were analyzed. In both, the phenotype of multiple exophytic melanocytic nevi was inherited in an autosomal dominant pattern (FIG. 1A; an extended pedigree of family 1 is shown in FIG. 2A). Affected family members, beginning in the third decade of life, develop numerous, slow-growing melanocytic nevi. Clinically, the nevi are skin-colored to reddish-brown, exophytic, dome-shaped to pedunculated, well-circumscribed papules with an average size of 5 mm (FIG. 1B). Several probands also had varying numbers of pigmented macular nevi, primarily on the trunk. The pathognomonic exophytic and non-pigmented nevi were primarily located on the lateral sides of the upper arms, the neck, the face and the scalp, with a few lesions present on the trunk. The number of nevi per patient varied markedly, ranging from five to over 50.

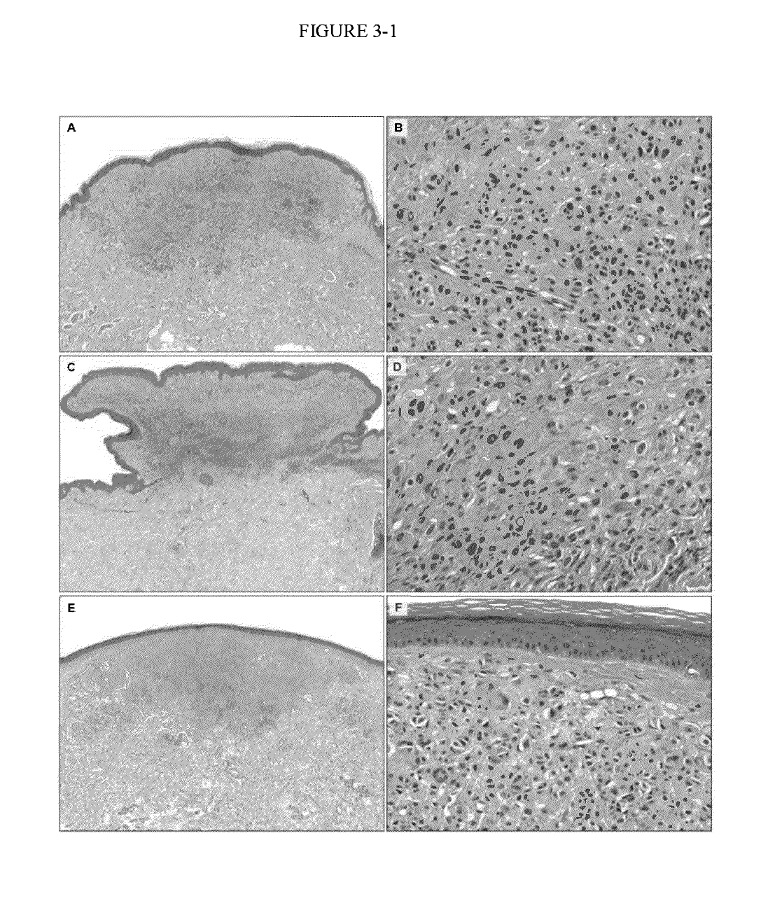

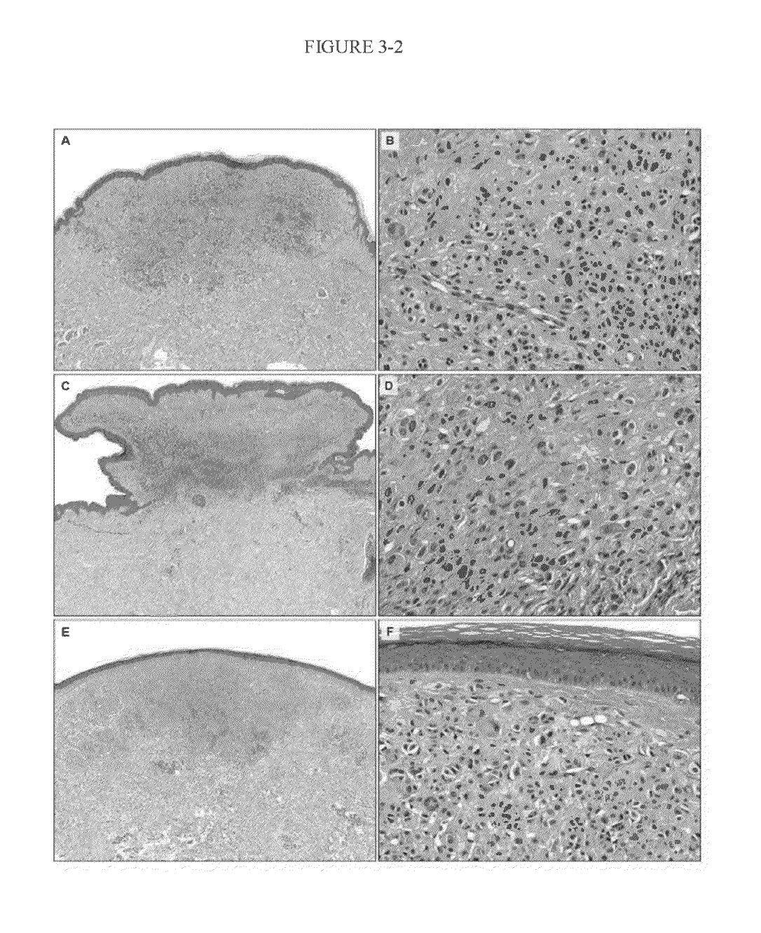

Histopathological examination revealed primarily dermal melanocytic nevi composed entirely, or predominantly, of epithelioid melanocytes with abundant amphophilic cytoplasm and nuclear pleomorphism (FIG. 1C and FIG. 3). The melanocytes often contained large, irregularly-shaped, vesicular nuclei with prominent nucleoli. Mitotic figures were scarce. In several cases, there were increased numbers of small blood vessels within the nevi, along with lymphangiectasia. Some nevi, interpreted as combined nevi, also contained an admixed component of smaller nevoid melanocytes (as seen in common acquired nevi). The cytological features of some of the cells were reminiscent of Spitz nevi; however, other characteristic features, such as junctional nests of melanocytes with clefts and epidermal hyperplasia, were consistently absent. In addition, 35 of 40 (88%) of the papular nevi showed v-raf murine sarcoma viral oncogene homolog B1 gene (BRAF) mutations, which are typically absent in Spitz nevi (Palmedo, et al., 2004). Many of the lesions showed imperfect maturation, i.e. the melanocytes did not significantly decrease in size with descent into the dermis. While none of the lesions demonstrated unequivocal histological evidence of malignancy, some exhibited high cellularity and substantial cytological atypia and were therefore considered to be of ‘uncertain malignant potential’. Due to this diagnostic uncertainty, two patients (II-4, II-8) in family 1 were managed as if they had melanoma. No cutaneous melanomas were diagnosed in family 1, but three probands in family 2 developed cutaneous melanoma at ages 38 (II-6, dorsum of the foot), 39 (III-3, scalp) and 50 (II-4, back) years, respectively. Patient II-6 developed a lymph node metastasis 13 years after excision of the primary tumor. One proband in family 1 (I-2) developed uveal melanoma, at age 72 years, and one in family 2 (II-2) developed uveal melanoma, at age 44 years.

Identification of a Candidate Region for a Tumor Suppressor Gene

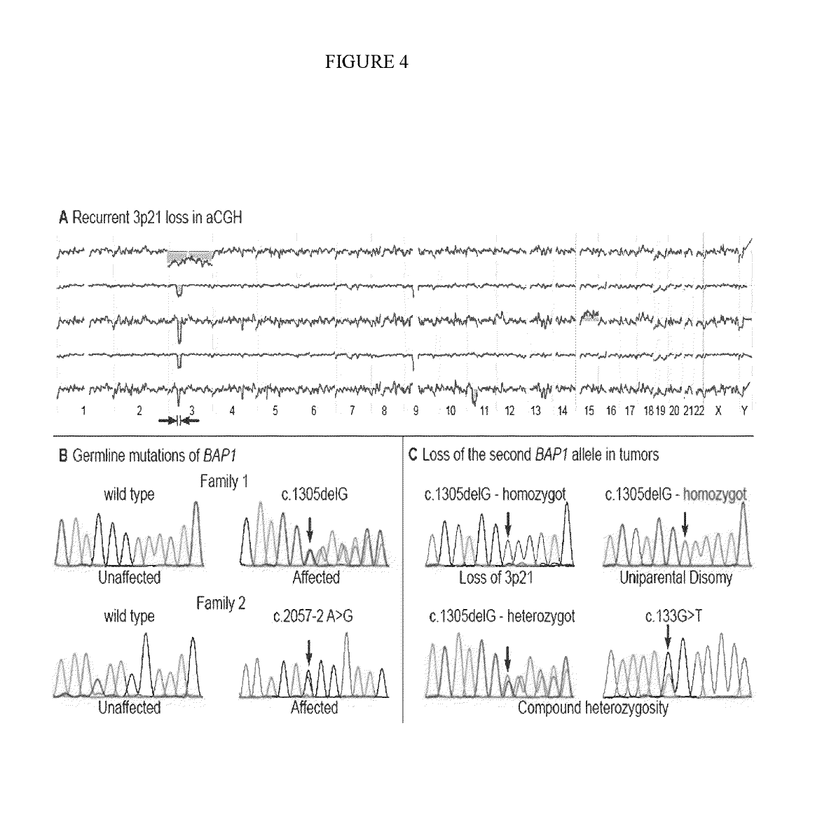

Twenty-two nevi from three probands in family 1 were analyzed with array-based comparative genomic hybridization (aCGH). Losses affecting the entire chromosome 3 or portions of the short arm of chromosome 3 were found in 50% of tumors (FIG. 4A). The smallest common region of deletion encompassed 5.8 megabases in 3p21, extending from base pair position 47,976,758 to 53,848,761 and encoding at least 150 genes (Human (Homo sapiens) Genome Browser Gateway, 2006).

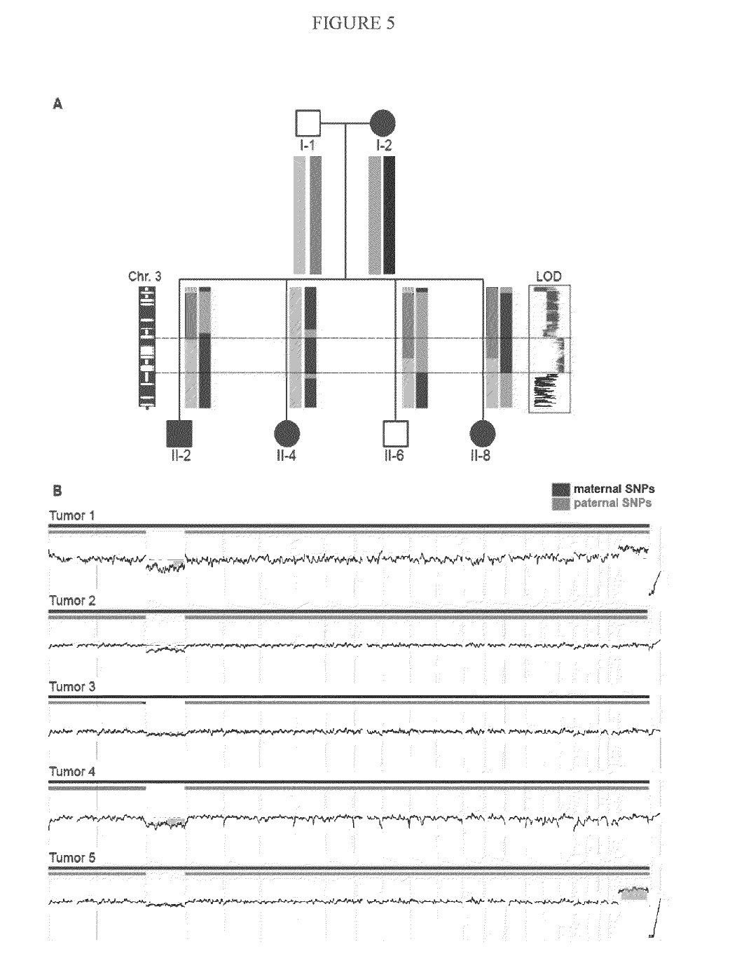

The frequent loss, in the nevi, of 3p21 suggested that such deletions may have been positively selected in the nevi because they eliminated the remaining wild type copy of a tumor suppressor gene. The haplotypes of six members of family 1 were, therefore, reconstructed with single-nucleotide polymorphism (SNP) arrays. Parametric linkage analysis showed that affected siblings in the second generation (II-2, II-4, II-8) inherited the same maternal (I-2) 3p allele, whereas the non-affected brother (II-6) inherited the other maternal 3p allele (FIG. 5A). SNP arrays of DNA extracted from the nevi demonstrated that, in tumors with chromosome 3 deletions, the paternal chromosome 3 was lost, while the (putatively mutated) maternal chromosome 3 was retained (FIG. 5B).

Identification of BAP1 as a Melanocytic Tumor Susceptibility Gene

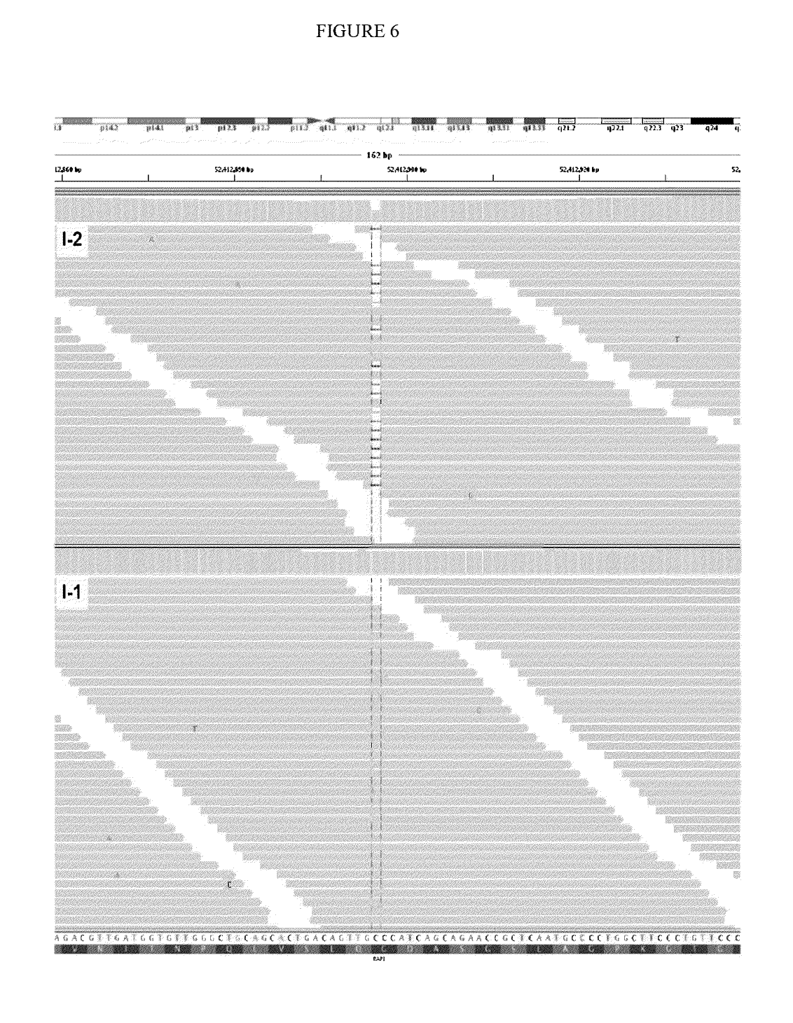

An in-solution hybrid capture technique, followed by massively parallel sequencing in four probands of family 1 (I-1, I-2, II-4, II-6), was used to identify mutated genes within the 3p21 region. This analysis revealed a deletion of a single guanidine in codon 435 of the BAP1 gene, leading to a frameshift and premature termination (c.1305delG; p.G1n436Asnfs*135) (FIG. 4B and FIG. 6). The mutation segregated with the phenotype of multiple papular nevi and was absent in unaffected family members (FIG. 2B).

The status of the second (paternal) BAP1 allele was assessed in 29 epithelioid nevi from family 1. Loss of heterozygosity (LOH), with elimination of the wild type allele, was found in all tumors in which aCGH had already shown loss of 3p. More subtle mutations of the second (paternal) BAP1 allele were found in nevi without 3p deletions, including two nonsense mutations resulting in premature protein termination, one frameshift mutation and one missense mutation (Table 1). In five additional cases without loss of the 3p21 region, the sequencing electropherogram showed marked decrease in residual wild type sequences, indicating loss of the wild type BAP1 allele through maternal uniparental disomy in the neoplastic cells (FIG. 4C). In summary, eight of eleven nevi that did not present evidence of BAP1 deletion by aCGH showed loss of the wild type allele by copy number-neutral mechanisms. The uveal melanoma in proband I-2 also showed a monosomy 3.

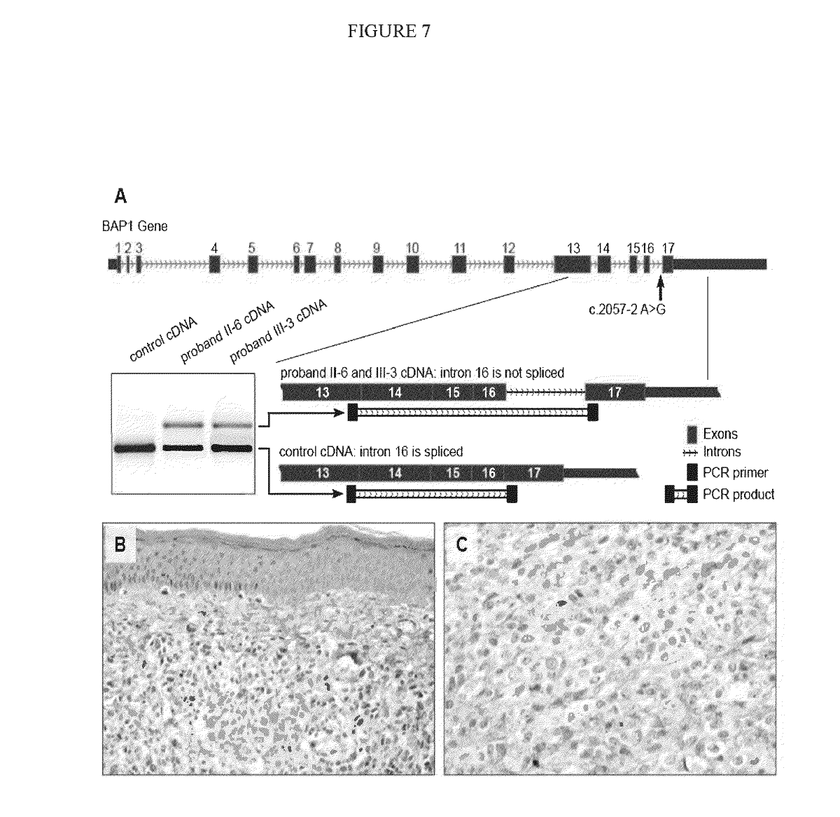

In family 2, a different germline mutation in BAP1 segregated with the phenotype of multiple exophytic nevi (FIG. 2B and FIG. 3C). The mutation, c.2057-2 A>G, removed an acceptor splice site at the last exon of BAP1, resulting in incorrect amino acids starting at position 687 and a premature stop codon at position 714 (p.Met687Glufs*28). Analysis of cDNA from two probands confirmed that the last intron was not removed by splicing (FIG. 7A). In nevi from family 2, inactivation of the remaining wild-type BAP1 allele by LOH was found in eight of twelve cases (Table 2). Interestingly, in the family 2 patient with metastasizing cutaneous melanoma, neither the primary tumor nor its metastasis showed loss of heterozygosity of BAP1. The uveal melanoma was not available for analysis.

Immunohistochemistry for BAP1 showed loss of nuclear staining in nevi from both families (FIG. 7B and 7C), which is consistent with the predicted consequences of the mutations found in BAP1, namely loss of the nuclear localization signal (NLS) preventing nuclear localization of BAP1 and/or loss of BAP1 expression by nonsense-mediated decay. To rule out similar mutations of BAP1 in the general population, available BAP1 sequence data from 629 individuals in the 1000 Genomes Project database (1000genomes.org/home) were reviewed. Filtering for sequence variants of BAP1, no truncating mutations were found, indicating that these mutations are infrequent in the general population.

TABLE 1
BAP1 status in melanocytic tumors of family 1.

| | | | | BAP1 | | Predicted functional | 3p21 loss | BRAF |
|---|---|---|---|---|---|---|---|---|
| Patient | # | Localization | Histology | Allele | Status | consequences | in aCGH | V600E |
| II-2 | 1 | upper back | combined | A | c.1305del | p.Gln436Asnfs*135 | | + |
| | | | | B | loss | | + | |
| | 2 | upper back | combined | A | c.1305del | p.Gln436Asnfs*135 | | wt |
| | | | | B | loss | | + | |
| | 3 | upper back | combined | A | c.1305del | p.Gln436Asnfs*135 | | + |
| | | | | B | loss | | + | |
| | 4 | upper arm | epithelioid | A | c.1305del | p.Gln436Asnfs*135 | | + |
| | | | | B | c.133G > T | p.Gly45* | − | |
| | 5 | upper arm | epithelioid | A | c.1305del | p.Gln436Asnfs*135 | | + |
| | | | | B | loss | | + | |
| | 6 | upper arm | epithelioid | A | c.1305del | p.Gln436Asnfs*135 | | + |
| | | | | B | UPD | | − | |
| | 7 | upper back | epithelioid | A | c.1305del | p.Gln436Asnfs*135 | | + |
| | | | | B | loss | | + | |
| | 8 | upper back | combined | A | c.1305del | p.Gln436Asnfs*135 | | + |
| | | | | B | UPD | | − | |
| | 9 | shoulder | epithelioid | A | c.1305del | p.Gln436Asnfs*135 | | + |
| | | | | B | c.1768C > T | p.Gln590* | − | |
| | 10 | upper arm | epithelioid | A | c.1305del | p.Gln436Asnfs*135 | | + |
| | | | | B | UPD | | − | |
| | 11 | neck | epithelioid | A | c.1305del | p.Gln436Asnfs*135 | | + |
| | | | | B | loss | | + | |
| | 12 | lower back | epithelioid | A | c.1305del | p.Gln436Asnfs*135 | | + |
| | | | | B | loss | | + | |
| | 13 | knee dorsal | epithelioid | A | c.1305del | p.Gln436Asnfs*135 | | + |
| | | | | B | loss | | + | |
| | 14 | knee dorsal | epithelioid | A | c.1305del | p.Gln436Asnfs*135 | | + |
| | | | | B | loss | | + | |
| | 15 | shoulder | epithelioid | A | c.1305del | p.Gln436Asnfs*135 | | + |
| | | | | B | loss/UPD | | n.a. | |
| | 16 | lower back | epithelioid | A | c.1305del | p.Gln436Asnfs*135 | | + |
| | | | | B | wt | | n.a. | |
| | 17 | knee ventral | epithelioid | A | c.1305del | p.Gln436Asnfs*135 | | wt |
| | | | | B | wt | | n.a. | |
| | 18 | lower leg | epithelioid | A | c.1305del | p.Gln436Asnfs*135 | | + |
| | | | | B | loss/UPD | | n.a. | |
| II-4 | 19 | shoulder | epithelioid | A | c.1305del | p.Gln436Asnfs*135 | | + |
| | | | | B | loss | | + | |
| | 20 | upper arm | epithelioid | A | c.1305del | p.Gln436Asnfs*135 | | + |
| | | | | B | wt | | − | |
| | 21 | lower arm | epithelioid | A | c.1305del | p.Gln436Asnfs*135 | | + |
| | | | | B | wt | | − | |
| | 22 | upper back | epithelioid | A | c.1305del | p.Gln436Asnfs*135 | | wt |
| | | | | B | UPD | | − | |
| | 23 | lower arm | epithelioid | A | c.1305del | p.Gln436Asnfs*135 | | + |
| | | | | B | loss/UPD | | n.a. | |
| | 24 | lower back | epithelioid | A | c.1305del | p.Gln436Asnfs*135 | | + |
| | | | | B | wt | | n.a. | |
| | 25 | ear | combined | A | c.1305del | p.Gln436Asnfs*135 | | + |
| | | | | B | c.1145dup | p.Arg383Profs*15 | n.a. | |
| II-8 | 26 | shoulder | epithelioid | A | C.1305del | p.Gln436Asnfs*135 | | + |
| | | | | B | loss | | + | |
| | 27 | upper arm | epithelioid | A | c.1305del | p.Gln436Asnfs*135 | | + |
| | | | | B | UPD | | − | |
| | 28 | upper arm | epithelioid | A | c.1305del | p.Gln436Asnfs*135 | | + |
| | | | | B | c.901G > A | p.Ala301Thr | − | |
| | 29 | upper arm | combined | A | c.1305del | p.Gln436Asnfs*135 | | + |
| | | | | B | UPD | | − | |
| I-2 | 30 | eye | uveal | A | c.1305del | p.Gln436Asnfs*135 | | wt |
| | | | melanoma | B | loss | | + | |

wt: wild type; n.a.: not available
epithelioid: melanocytic tumor composed of cells with epithelioid morphology.
combined: melanocytic tumor composed predominately of epithelioid cells and some admixed nevoid cells.
UPD: uniparental disomy of maternal sequences, as assessed by markedly suppressed residual wild type sequences in the electropherograms, but no loss of 3p21 in aCGH.
UPD/loss: analysis of the electropherograms indicated a loss of the wild type allele, but differentiation between UPD and loss of 3p21 was not possible, because aCGH was not performed.
+: loss of the 3p21 region or BRAF V600E mutation, −: no loss of 3p21

TABLE 2
BAP1 status in melanocytic tumors of family 2.

| | | | | BAP1 | | Predicted functional | 3p21 | BRAF |
|---|---|---|---|---|---|---|---|---|
| Patient | # | Localization | Histology | Allele | Status | consequences | loss in | V600E |
| III-3 | 1 | neck | combined | A | c.2057-2 A > G | p.Met687Glufs*28 | | + |
| | | | | B | loss | | + | |
| | 2 | occipital | epithelioid | A | c.2057-2 A > G | p.Met687Glufs*28 | | + |
| | | | | B | loss/UPD | | n.a. | |
| | 3 | upper back | epithelioid | A | c.2057-2 A > G | p.Met687Glufs*28 | | + |
| | | | | B | loss/UPD | | n.a. | |
| | 4 | upper back | epithelioid | A | c.2057-2 A > G | p.Met687Glufs*28 | | + |
| | | | | B | loss/UPD | | n.a. | |
| | 5 | upper arm | combined | A | c.2057-2 A > G | p.Met687Glufs*28 | | + |
| | | | | B | loss | | + | |
| | 6 | upper arm | epithelioid | A | c.2057-2 A > G | p.Met687Glufs*28 | | + |
| | | | | B | UPD | | − | |
| | 7 | shoulder | epithelioid | A | c.2057-2 A > G | p.Met687Glufs*28 | | + |
| | | | | B | wt | | n.a. | |
| III-2 | 8 | lower arm | MELTUMP | A | c.2057-2 A > G | p.Met687Glufs*28 | | + |
| | | | | B | loss | | + | |
| | 9 | ear | epithelioid | A | c.2057-2 A > G | p.Met687Glufs*28 | | + |
| | | | | B | loss/UPD | | n.a. | |
| II-4 | 10 | lower back | epithelioid | A | c.2057-2 A > G | p.Met687Glufs*28 | | wt |
| | | | | B | wt | | n.a. | |
| | 11 | lower leg | combined | A | c.2057-2 A > G | p.Met687Glufs*28 | | wt |
| | | | | B | wt | | n.a. | |
| | 12 | lower back | epithelioid | A | c.2057-2A > G | p.Met687Glufs*28 | | + |
| | | | | B | wt | | n.a. | |
| | 13 | upper arm | MELTUMP | A | c.2057-2A > G | p.Met687Glufs*28 | | + |
| | | | | B | loss/UPD | | n.a. | |
| | 14 | upper back | cutaneous | A | c.2057-2A > G | p.Met687Glufs*28 | | wt |
| | | | melanoma | B | loss/UPD | | n.a. | |
| II-6 | 15 | dorsum of | cutaneous | A | c.2057-2 A > G | p.Met687Glufs*28 | | + |
| | | the foot | melanoma | B | wt | | − | |
| | 16 | inguinal | lymphnode | A | c.2057-2 A > G | p.Met687Glufs*28 | | + |
| | | | metastasis | B | wt | | + | |
| II-2 | 17 | eye | uveal | A | c.2057-2 A > G | p.Met687Glufs*28 | | wt |
| | | | melanoma | B | loss | | + | |

wt: wild type; n.a.: not available;
epithelioid: melanocytic tumor composed of epithelioid cells;
combined: melanocytic tumor composed predominately of epithelioid cells and some admixed nevoid cells;
MELTUMP: melanocytic tumor of uncertain malignant potential;
UPD: uniparental disomy of maternal sequences, as assessed by markedly suppressed residual wild type sequences in the electropherograms, but no loss of 3p21 in aCGH;
UPD/loss: analysis of the electropherograms indicates a loss of the wild type allele, but differentiation between UPD and loss of 3p21 was not possible, because aCGH was not performed;
+: loss of the 3p21 region or BRAF V600E mutation; −: no loss of 3p21.

BAP1 Mutations in Sporadic Melanocytic Tumors

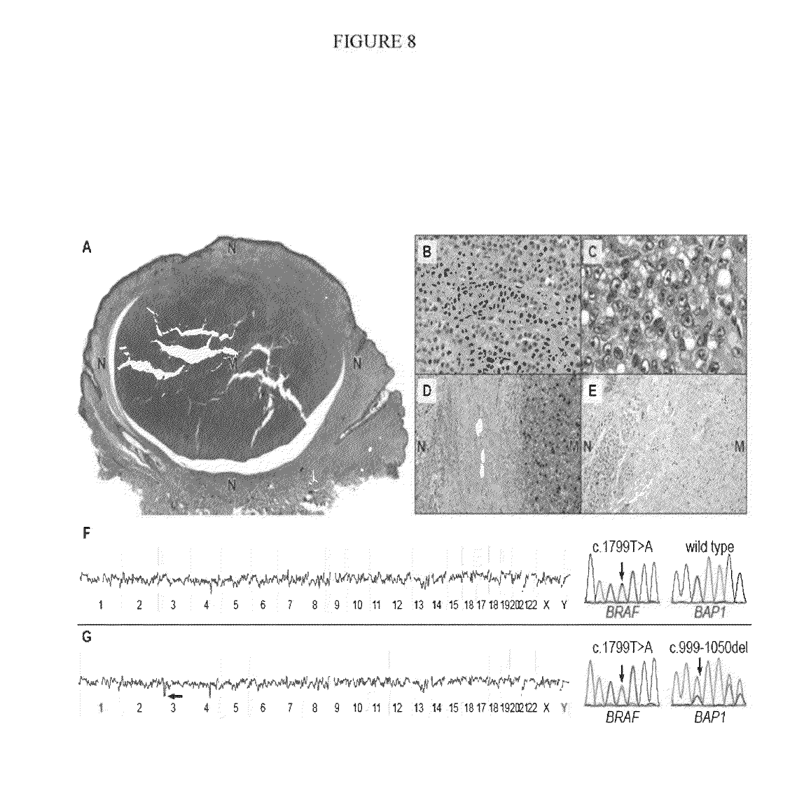

The findings in the two families analyzed suggested that BAP1 mutations strongly predispose to cutaneous melanocytic proliferations composed of epithelioid melanocytes with benign or borderline histopathological features. While several hundred papular nevi were present among the probands in each family, the number of melanomas was substantially lower. To investigate the role of BAP1 in melanoma, the prevalence of BAP1 mutations in melanomas without known family history was analyzed. Consistent with the findings of a recent study (Harbour, et al., 2010), BAP1 mutations were found in 13 of 33 (40%) primary uveal melanomas (Table 3). In contrast, only three BAP1 mutations (two frameshift mutations and one missense mutation) (Table 4) were found in 60 sporadic primary cutaneous melanomas (comprising 15 cases in each of four groups: acral and mucosal melanomas, and melanomas on skin with or without chronic sun-induced damage). One of these cases, presented in FIG. 8, was a melanoma arising in a nevus and illustrates how loss of BAP1 may play a critical role in the progression from nevus to melanoma in some cases. While the nevus cells showed nuclear expression of BAP1 by immunohistochemistry, the melanoma cells did not. Concordantly, the microdissected nevus component showed wild type BAP1 sequence and normal DNA copy number by aCGH (FIG. 8F), while the melanoma component harbored a BAP1 frameshift mutation as well as an interstitial deletion of chromosome 3p21 spanning the BAP1 locus (FIG. 8G). Both the nevus and the melanoma component showed the same BRAF^{V600E} mutation.

TABLE 3
BAP1 Status in 33 Primary Uveal Melanomas

| | BAP1 | Predicted protein | Chrom. Aberrations detected by aCGH | | | | GNAQ/GNA11 |
|---|---|---|---|---|---|---|---|
| Sample | mutations | sequence | 3 | 1p | 6q | 8q | status |
| 1 | c.1153C > T | p.(Arg385*) | loss | loss | — | gain | GNAQ Q209P |
| 2 | c.219del | p.(Asp73Glufs*5) | loss | loss | — | gain | GNA11 Q209L |
| 3 | c.58G > T | p.(Glu20*) | loss | loss | loss | — | GNA11 Q209L |
| 4 | c.643del | p.(Glu212Serfs*19) | loss | — | — | gain | GNAQ Q209L |
| 5 | c.915dup | p.(Glu306*) | loss | — | — | gain | GNA11 Q209L |
| 6 | c.812_821del | p.(Ile271Thrfs*61) | loss | — | — | gain | GNAQ Q209P |
| 7 | c.226_239del | p.(Ile76Valfs*45) | loss | loss | — | — | GNA11 Q209L |
| 8 | c.6dup | p.(Lys3*) | loss | — | — | gain | GNA11 Q209L |
| 9 | c.993del | p.(Lys331Asnfs*4) | loss | loss | loss | gain | GNAQ Q209P |
| 10 | c.243C > A | p.(Phe81Leu) | loss | — | — | gain | GNAQ Q209P |
| 11 | c.172_179del | p.(Ser58Lysfs*8) | loss | — | loss | gain | GNAQ Q209P |
| | c.139A > T | p.(Ile47Phe) | | | | | |
| 12 | c.188C > G | p.(Ser63Cys) | loss | — | loss | gain | GNA11 Q209L |
| 13 | wt | wt | loss | loss | — | — | GNAQ Q209P |
| 14 | wt | wt | loss | — | — | gain | GNAQ Q209P |
| 15 | wt | wt | loss | — | loss | gain | GNA11 Q209L |
| 16 | wt | wt | partial loss | loss | loss | gain | wt |
| 17 | c.1379C > G | p.(Ser460*) | partial loss | — | — | gain | GNAQ Q209P |
| 18 | wt | wt | partial loss | — | — | gain | GNA11 Q209L |
| 19 | wt | wt | partial loss | — | loss | — | GNAQ Q209P |
| 20 | wt | wt | partial loss | loss | loss | gain | GNAQ Q209P |
| 21 | wt | wt | partial loss | — | — | — | GNAQ Q209P |
| 22 | wt | wt | — | — | — | — | GNAQ Q209P |
| 23 | wt | wt | — | — | — | — | GNA11 Q209L |
| 24 | wt | wt | — | — | — | — | wt |
| 25 | wt | wt | — | — | — | gain | GNA11 Q209L |
| 26 | wt | wt | — | — | — | — | GNAQ Q209P |
| 27 | wt | wt | — | — | — | gain | GNAQ Q209P |
| 28 | wt | wt | — | loss | — | — | GNAQ Q209P |
| 29 | wt | wt | — | — | — | gain | GNA11 Q209L |
| 30 | wt | wt | — | — | — | — | GNAQ Q209R |
| 31 | wt | wt | — | — | — | — | GNAQ Q209L |
| 32 | wt | wt | — | loss | loss | gain | GNAQ Q209P |
| 33 | c.1002A > G | p.(=) | — | — | — | — | GNAQ Q209P |

TABLE 4
BAP1 Mutations Detected in 60 Sporadic Cutaneous Melanomas

| Sam- | | | | Predicted functional |
|---|---|---|---|---|
| ple | Allele | Status | Mutation | consequences |
| 1 | A | Mutation | c.999_1046del | p.(Val335Profs * 10) |
| | B | loss | | |
| 2 | A | Mutation | c.278_281delCTCA | p.(Thr93Metfs * 4) |
| | B | loss | | |
| 3 | A | Mutation | c.2090C > G, | p.(Ser697Cys) |
| | B | wt | | |

Materials and Methods

Accession Numbers

The mutation nomenclature is based on the following National Center for Biotechnology information (NCB1) reference sequences: BAP1 cDNA, NM_{—}004656.2; BAP1 protein, NP_{—}004647.1; BRAF cDNA, NM_{—}004333.4; BRAF protein, NP_{—}004324.2; GATAQ cDNA, NM_{—}002072.3; GNAQ protein, NP_{—}002063.2; GNA11 cDNA NM_{—}002067.2; GNA11 protein, NP_{—}002058.2.

Subjects

Two families in which several members developed multiple distinct melanocytic nevi (FIG. 1A) were studied. From family 1, blood was collected from six probands (I-1, I-2, II-2, II-4, II-6, II-8), 29 melanocytic nevi from three probands (II-2, n=18; II-4, n=7; II-8, n=4), and one uveal melanoma from proband I-2. From family 2, blood was collected from nine probands (II-3 to 6, III-1 to 5), twelve melanocytic nevi from three probands (Ii-4, n=3; III-2, n=2; III-4, n=7), and one cutaneous melanoma from proband II-6 (primary tumor and lymph node metastasis). The study was approved by the Ethics Committees of the Medical University of Graz and Memorial Sloan-Kettering Cancer Center, and written consent was obtained from all participating family members.

Tissues

Archival, paraffin-embedded biopsies were retrieved from the Department of Dermatology, Medical University of Graz, Austria and from the Department of Pathology, Memorial Sloan-Kettering Cancer Center, New York under the approval of their respective Institutional Review Boards.

Histopathologic Evaluation and Immunohistochemistry

Excised skin lesions were fixed in 4% neutral-buffered formalin. The fixed tissues were processed using routine histologic methods and embedded in paraffin. 5 μm-thick sections were cut from the paraffin blocks and stained with hematoxylin-eosin. Immunohistochemistry using an antibody against BRCA1 -associated protein-1 [BAP1 (C-4), Santa Cruz Biotechnology, Santa Cruz, CA] was performed on tissue sections using standard methods.

DNA Extraction

Tumor-bearing tissue was manually microdissected from sections of archival, paraffin-embedded samples of nevi and melanomas. In the case of small tumors, tumor-bearing tissue was microdissected using a PALM Laser Microdissection and Pressure Catapulting system (Carl Zeiss, Germany). DNA was extracted from the microdissected tissue and purified using the QIAamp DNA FFPE Tissue Kit (Qiagen, Hilden, Germany) or the chemagic DNA Tissue Kit (Chemagen, Baesweiler, Germany) according to the manufacturers' instructions.

Array-based Comparative Genomic Hybridization (aCGH)

DNA samples were labeled using a Bioprime Array CGH Genomic Labeling Kit (Invitrogen, Carlsbad, Calif.) according to the manufacturer's instructions. Briefly, 250 ng of amplified test and reference (Promega, Madison, Wis.) DNA were differentially labeled with dCTP-Cy5 and dCTP-Cy3, respectively (GE Healthcare, Piscataway, N.J.). Genome-wide analysis of DNA copy number changes was conducted using an oligonucleotide array containing 60,000 probes (Agilent, Santa Clara, Calif.) according to the manufacturer's protocol version 6.0. Slides were scanned using Agilent's microarray scanner G2505B and analyzed using Agilent Feature Extraction and DNA Workbench software 5.0.14.

DNA Sequencing

The exonic regions of BAP1 were divided into amplicons of 300 base pairs (bp) or less (Table 5). Specific primers were designed using primer 3 and tagged with an M13 tail to facilitate Sanger sequencing. The PCR reaction conditions were 0.25 mM deoxyribonucleotide triphosphates (dNTPs), 0.4× bovine serum albumin (BSA) (New England Biolabs, Ipswich, Mass.), 1 U Hotstar Taq (Qiagen), 1× Hotstar Taq buffer (Qiagen), and 0.5 μM each primer (as shown in Table 5). PCR consisted of 35 cycles of 95° C. (30 seconds), 58° C. (1 minute), and 72° C. (1 minute) after initial denaturation at 94° C. for 15 minutes. PCR reaction products were purified using QIAquick PCR Purification kit (Qiagen) and then used as templates for sequencing reactions using Big Dye v3.1 (Applied Biosystems, Carlsbad, Calif.); sequencing was performed in both directions. Dye terminators were removed using the Agencourt CleanSEQ kit (Beckman Coulter, Danvers, Mass.), and subsequent products were run on the ABI PRISM 3730x1 (Applied Biosystems). Mutations were identified by using Sequencher Software (genecodes.com) and only considered when variants were called in reads in both directions.

TABLE 5
Primers for BAP1 Sequencing

| Exon | Forward | | Reverse | |
|---|---|---|---|---|
| DNA | GGAGGGCCTGGACATGG | (SEQ ID NO: 3) | ATGAGTGAGGGCGCAGG | (SEQ ID NO: 4) |
| Primers | | | | |
| Exon 1-2 | | | | |
| Exon 3 | GGGCTGTCCTTCCCTACTG | (SEQ ID NO: 5) | CCTGTTCTCTGGGACCTTCC | (SEQ ID NO: 6) |
| Exon 4 | ATTGTCTTCTCCCCTTTGGC | (SEQ ID NO: 7) | AACATGGCAGCATCCCAC | (SEQ ID NO: 8) |
| Exon 5 | GTGAGGGGTGCTGTGTATGG | (SEQ ID NO: 9) | AGTTGGCTGTGAGCCAGG | (SEQ ID NO: 10) |
| Exon 6 | TTTGCCTTCCACCCATAGTC | (SEQ ID NO: 11) | ACTCCCACCCCACATCAG | (SEQ ID NO: 12) |
| Exon 7 | GCTGATGTGGGGTGGGAG | (SEQ ID NO: 13) | GGAGGTAGGCAGAGACACCC | (SEQ ID NO: 14) |
| Exon 8 | ACTCAGGGTTTCCTTCTCGC | (SEQ ID NO: 15) | TCTGTCCCTCCCAAAGTAGG | (SEQ ID NO: 16) |
| Exon 9 | CTCAACCTGATGGCGGG | (SEQ ID NO: 17) | AATGCAGGGAGGGTTGG | (SEQ ID NO: 18) |
| Exon 10 | CGGGTCTACCCTTTCTCCTC | (SEQ ID NO: 19) | AGACATTAGCGGGTGGCTC | (SEQ ID NO: 20) |
| Exon 11 | GGAGGTCCTGCCTGTGTTC | (SEQ ID NO: 21) | GGAACCACATGGGAAAATTG | (SEQ ID NO: 22) |
| Exon 12 | CCGAGCAGCACTTGTTTG | (SEQ ID NO: 23) | GATCCGAAGCACCTAGAACC | (SEQ ID NO: 24) |
| Exon 13_1 | GCCCGTTCCCTTGCTTC | (SEQ ID NO: 25) | GTGAGGGCTGCGAGTGTG | (SEQ ID NO: 26) |
| Exon 13_2 | CCTCTCAATTCCTCTGTCCATC | (SEQ ID NO: 27) | GCAGGCTGTCATCCTCTCC | (SEQ ID NO: 28) |
| Exon 13_3 | CTATCCGCTCAGCCAACC | (SEQ ID NO: 29) | TCCCTCCTCCCTCCTGG | (SEQ ID NO: 30) |
| Exon 14 | CCACAAAGTGTCCTGCACTC | (SEQ ID NO: 31) | AGCTCAGGCCTTACCCTCTG | (SEQ ID NO: 32) |
| Exon 15 | GTGGGGCTTTGTTGCTG | (SEQ ID NO: 33) | CAGTGGACCTCGGGAGAG | (SEQ ID NO: 34) |
| Exon 16 | AGATTGGCTCCAGTGCTCTC | (SEQ ID NO: 35) | AGCAGGGCATTCCAGTTAAG | (SEQ ID NO: 36) |
| Exon 17 | ATGAGAGCCTCAGCTCCTGG | (SEQ ID NO: 37) | CACACGGCAAGAGTGGG | (SEQ ID NO: 38) |
| cDNA | ATCTGGGTCCTGTCATCAGC | (SEQ ID NO: 39) | ACGGAGATGTTCTGCTCCAC | (SEQ ID NO: 40) |
| Primers | | | | |
| Exon 13-17 | | | | |

RNA Analysis

To detect the effect of the splice site mutation, RNA was isolated from whole blood of affected family members and normal controls. The isolated RNA was reverse transcribed using Qiagen's Omniscript Reverse Transcriptase and subsequently amplified using cDNA specific primers for BAP1 (Table 5). PCR consisted of 35 cycles of 95° C. (30 seconds), 58° C. (1 minute), and 72° C. (1 minute) after initial denaturation at 94° C. for 15 minutes. PCR products were electrophoretically separated on a 1% agarose gel. Bands were cut out, purified using gel purification (Promega Wizard SV-Gel and PCR cleanup system), and sequenced on a 3130x1 capillary genetic analyzer (Applied Biosystems).

Single-nucleotide Polymorphism (SNP) Arrays

DNA from four affected (I-2, II-2, II-4, II-8) and two unaffected (I-1, II-6) individuals from family 1 were analyzed with the Affymetrix (Santa Clara, Calif.) GeneChip Mapping 500K array NspI chip. In accordance with the manufacturer's instructions (Affymetrix Mapping 500K Assay Manual), the arrays were scanned with a GeneChip Scanner, and the data were analyzed with GeneChip Operating Software (GCOS) and GeneChip Genotyping Analysis Software (GTYPE) software (v3.0.2) for generation of SNP allele calls. Haplotyping and parametric linkage analysis were performed with the DNA-Chip Analyzer (dChip) software (dchip.org) assuming an autosomal dominant trait.

Target Enrichment and SOLiD Sequencing

DNA capture was performed on 3 μg of high quality genomic DNA using a custom SureSelect Target Enrichment kit according to the protocol provided by Agilent. The baits library for SureSelect Target Enrichment was designed in Agilent's eArray. Chromosome 3p21 enriched DNA libraries of four probands (I-1, I-2, II-4, II-6) of family 1 were sequenced on a SOLiD 4 system according to the protocol provided by Applied Biosystems, generating an average of 73,096,487 reads per sample with a read length of 50 bp. On average, 78% of the targeted region was covered at 400×.

To detect small insertion/deletion events, the SOLiD data were processed through the following pipeline: The reads were mapped using the Burrows-Wheeler Alignment Tool (v0.5.8c) (Li & Durbin, 2009) with default options except for the colorspace switch. The mapped BAM file was then processed to remove duplicated reads and realigned with the IndelRealigner from the Genome Analysis Toolkit (GATK), which does a more careful alignment of reads around putative insertions/deletions (McKenna, et al., 2010) (DePristo, et al., 2011). The realigned BAM file was converted to a pileup with SAMtools, and insertion/deletion variants were called with the VarScan (v2.2) program.

### REFERENCES

Buchhagen, D. L., Qiu, L., & Etkind, P. (1994). Homozygous Deletion, Rearrangement and Hypermethylation Implicate Chromosome Region 3p14.3-3p21.3 in Sporadic Breast-Cancer Development. International Journal of Cancer 57(4), 473-479.
de Snoo, F. A., & Hayward, N. K. (2005). Cutaneous Melanoma Susceptibility and Progression Genes. Cancer Letters 23(2), 153-186.
DePristo, M., Banks, E., Poplin, R., Garimella, K., Maguire, J., Hartl, C., et al. (2011). A Framework for Variation Discovery and Genotyping Using Next-generation DNA Sequencing Data. Nature Genetics 43(5), 491-498.
Garber, J. E., & Offit, K. (2005). Hereditary Cancer Predisposition Syndromes. Journal of Clinical Oncology 23(2), 276-292.
Harbour, J. W., Onken, M. D., Roberson, E. D., Duan, S., Cao, L., Worley, L. A., et al. (2010). Frequent Mutation of BAP1 in Metastasizing Uveal Melanomas. Science 330 (6009), 1410-1413. Human (Homo sapiens) Genome Browser Gateway, NCBI36/hg18 Assembly. (2006, March). Retrieved from UCSC Genome Bioinformatics: genome.ucsc.edu/cgi-bin/hgGateway?db=hg18 Jensen, D. E., Proctor, M., Marquis, S. T., Gardner, H. P., Ha, S. I., Chodosh, L. A., et al. (1998). BAP1: A Novel Ubiquitin Hydrolase Which Binds to the BRCA1 RING Finger and Enhances BRCA1-mediated Cell Growth Suppression. Oncogene 16(9), 1097-1112.
Knudson, A. G. (1971). Mutation and Cancer: Statistical Study of Retinoblastoma. Proceedings of the National Academy of Sciences 68(4), 820-823.
Li, H., & Durbin, R. (2009). Fast and Accurate Short Read Alignment with Burrows-Wheeler Transform Bioinformatics 25(14), 1754-1760.
Matsuoka, S., Ballif, B. A., Smogorzewska, A., McDonald, E. R., Hurov, K. E., Luo, J., et al. (2007). ATM and ATR Substrate Analysis Reveals Extensive Protein Networks Responsive to DNA Damage. Science 316(5828), 1160-1166.
McKenna, A., Hanna, M., Banks, E., Sivachenko, A., Cibulskis, K., Kernytsky, A., et al. (2010). The Genome Analysis Toolkit: A MapReduce Framework for Analyzing Next-generation DNA Sequencing Data. Genome Research 20(9), 1297-1303.
Palmedo, G., Hantschke, M., Rütten, A., Mentzel, T., Hügel, H., Flaig, M. J., et al. (2004). The T1796A Mutation of the BRAF Gene is Absent in Spitz Nevi. Journal of Cutaneous Pathology 31(3), 266-270.
Scheuermann, J. C., de Ayala Alonso, A. G., Oktaba, K., Ly-Hartig, N., McGinty, R. K., Fraterman, S., et al. (2010). Histone H2A Deubiquitinase Activity of the Polycomb Repressive complex PR-DUB. Nature 465(7295), 243-247.
Stokes, M. P., Rush, J., MacNeill, J., Ren, J. M., Sprott, K., Nardone, J., et al. (2007). Profiling of UV-induced ATM/ATR Signaling Pathways. Proceedings of the National Academy of Sciences 104(50), 19855-19860.
Ventii, K. H., Devi, N. S., Friedrich, K. L., Chernova, T. A., Tighiouart, M., Van Meir, E. G., et al. (2008). BRCA1-Associated Protein-1 Is a Tumor Suppressor that Requires Deubiquitinating Activity and Nuclear Localization. Cancer Research 68(17), 6953-6962.

### Wood, L. D., Parsons, D. W., Jones, S., Lin, J., Sjoblom, T., Leary, R. J., et al. (2007). The Genomic

Landscapes of Human Breast and Colorectal Cancers. Science 318(5853), 1108-1113.

### EXAMPLE 2

### A Distinct Subset of Atypical Spitz Tumors is Characterized by BRAF Mutation and Loss of BAP1 Expression

We recently reported that germline mutations in BAP1 cause a familial tumor syndrome characterized by high penetrance for melanocytic tumors with distinctive clinical and histologic features (see Example 1). Melanocytic neoplasms in affected individuals harbored BRAF mutations, showed loss of BAP1 expression, and histologically resembled so-called “atypical Spitz tumors” (ASTs). ASTs are an ill-defined and probably heterogenous group of melanocytic tumors that display histologic features seen in both Spitz nevi and melanomas. Their biologic behavior cannot be reliably predicted. Based on the histologic similarities of the familial tumors and ASTs, we hypothesized that a subset of ASTs might harbor genetic alterations seen in the familial tumors.

To address this hypothesis, we analyzed 32 sporadic ASTs for BRAF mutations, and for BAP1 expression. To determine the utility of immunohistochemistry to assess BAP1 status, we analyzed samples from 42 patients with the above syndrome as well as 46 melanocytic nevi and found that loss of BAP1 expression was restricted to patients with the syndrome. Nine (28%) sporadic ASTs showed loss of BAP1 expression, of which of 8 (89%) had concomitant BRAF mutations. Only one of the BAP1-positive ASTs (4%) had a BRAF mutation (p<0.0001). BRAF mutated, BAP1-negative tumors were primarily located in the dermis, and were composed entirely or predominantly of epithelioid melanocytes with abundant amphophilic cytoplasm and well-defined cytoplasmic borders. Nuclei were commonly vesicular, and exhibited substantial pleomorphism and conspicuous nucleoli. The combination of BRAF mutation and loss of nuclear BAP1 expression thus characterizes a subset of ASTs with distinctive histologic features. The typical morphology of these tumors and BAP1 immunohistochemistry provide pathologic clues that will enable accurate identification of this subset. Future studies are necessary to determine whether this subset has a predictable clinical behavior.

### Introduction

Since the recognition of Spitz nevi, pathologists have increasingly identified a group of melanocytic tumors that exhibit histologic features overlapping those of Spitz nevi and melanomas. These tumors are often referred to as ‘Atypical Spitz tumors’ (ASTs) and are likely to represent a heterogeneous group of tumors that share some morphologic similarities. ASTs may cause diagnostic problems because their unequivocal histologic separation into Spitz nevi and spitzoid melanoma is not always possible, as demonstrated by a significant lack of interobserver agreement, even among experts.^{1,2,5}

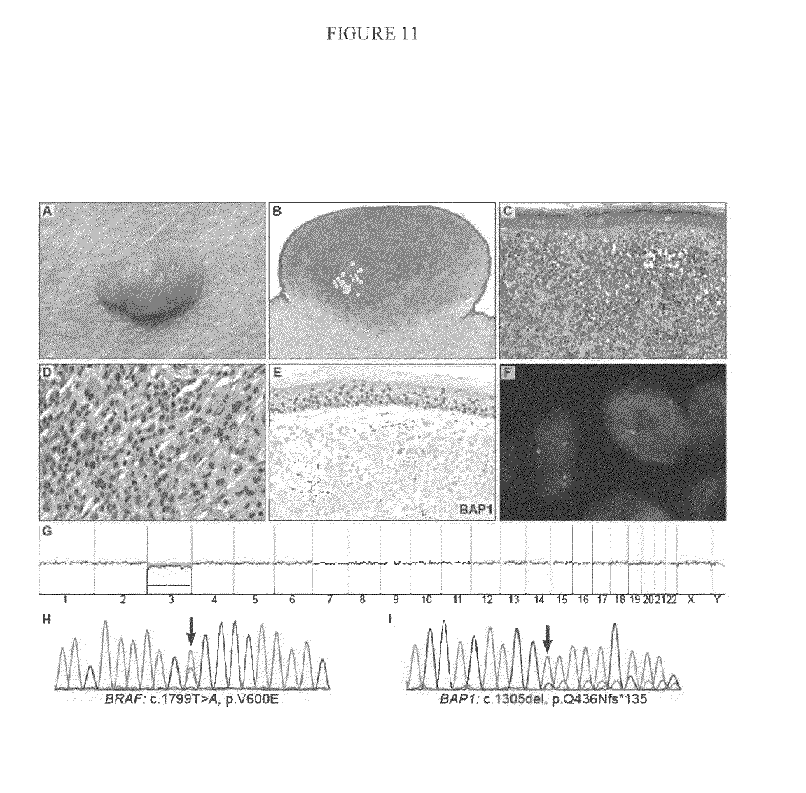

Recently, we described an autosomal dominant tumor syndrome caused by inactivating germline mutations of the BAP1 gene.⁽¹⁶⁾ Affected individuals had multiple cutaneous spitzoid melanocytic neoplasms and were predisposed to increased risk of developing cutaneous and uveal melanoma.(¹⁶) The characteristic cutaneous melanocytic tumors were skin-colored papules or nodules. Histologically, they were composed of dermal aggregates of epithelioid melanocytes with abundant amphophilic cytoplasm, pleomorphic vesicular nuclei, and conspicuous nucleoli. The majority of the tumors lost the remaining wild-type BAP1 allele by various somatic alterations and lacked immunohistochemical expression of BAP1 (FIG. 11).⁽¹⁶⁾

Although many of the cytologic features of the tumor cells were reminiscent of epithelioid cells of Spitz nevi, other features characteristically present in Spitz nevi (e.g. clefting around junctional melanocytic nests, spindle-shaped melanocytes, epidermal hyperplasia, hypergranulosis, and Kamino bodies)⁶ were consistently absent. Furthermore, the vast majority of the familial BAP1-negative neoplasms showed BRAF^{V600E} mutations¹⁶, which are typically absent in Spitz nevi.⁹ The characteristic morphology and distinct genetic aberrations in the familial melanocytic tumors suggested that these neoplasms constitute a distinct category of melanocytic tumors. In this study, we analyzed a series of ASTs with no known family history to determine whether histologic features, BAP1 expression, and mutation status of BRAF or HRAS are helpful in sub-classifying this challenging category of melanocytic neoplasms.

### Methods

Specimens

The study was approved by the Ethics Committees of the Medical University of Graz, Graz, Austria and the Memorial Sloan-Kettering Cancer Center, New York, USA, and was conducted according to the Declaration of Helsinki Specimens were fixed in 4% buffered formalin, routinely processed, and embedded in paraffin. 4 μm-thick sections were stained routinely with hematoxylin and eosin for histologic evaluation.

Two sets of cases were collected. The evaluation set for BAP1 immunohistochemistry consisted of 46 positive controls (29 common acquired nevi and 17 classical Spitz nevi showing no BAP1 mutations) and 42 negative controls (epithelioid melanocytic tumors from 2 families with BAP1 germline mutations; 29 tumors from family 1 and 13 tumors from family 2, as described in ref 16). The independent test set of 32 sporadic ASTs with epithelioid cytomorphology was collected

| | | Pathologic | | | Thickness | | | |
|---|---|---|---|---|---|---|---|---|
| [symbol] | Site | diagnosis | Architecture | Details | (mm) | Mitoses | TILs | [symbol] |
| [symbol] | Anterior | AST | Polypoid and | Sheets and nests of epithelioid cells with abundant | 4.5 | Absent | +++ | [symbol] |
| | shoulder | | compound, | amphophilic cytoplasm, well-defined | | | | |
| | | | predominantly | cytoplasmic borders and vesicular nuclei | | | | |
| | | | dermal | exhibiting moderate pleomorphism. Scattered | | | | |
| | | | | giant/multinucleate tumor cells. | | | | |
| [symbol] | Back | AST | Dermal | Sheets and nests of large epithelioid cells with | 2.5 | Absent | +++ | [symbol] |
| | | | | abundant amphophilic cytoplasm, well- | | | | |
| | | | | defined cytoplasmic borders and very | | | | |
| | | | | pleomorphic round/oval | | | | |
| | | | | vesicular nuclei with conspicuous nucleoli. | | | | |
| | | | | Scattered giant/multinucleate tumor cells. | | | | |
| [symbol] | Scalp | AST | Compound, | Sheets and nests of epithelioid cells with abundant | 5.0 | 1/mm2, | ++ | [symbol] |
| | | | predominantly | amphophilic cytoplasm, well-defined | | superficial | | |
| | | | dermal, with | cytoplasmic borders and vesicular nuclei | | | | |
| | | | congenital nevus- | exhibiting moderate pleomorphism. Scattered | | | | |
| | | | like pattern | giant/multinucleate tumor cells. | | | | |

[symbol]

indicates data missing or illegible when filed

from the diagnostic files and consultation cases of the authors. The sequencing and clinical data of the evaluation set and of two of the ASTs (cases 8 and 9) were published previously.¹⁶ Available clinical and pathologic characteristics of the tumors are summarized in Table 6.

TABLE 6
Clinical and pathologic features of sporadic atypical Spitz tumors with epithelioid morphology

| | | | | | | | | | |
|---|---|---|---|---|---|---|---|---|---|
| [symbol] | [symbol] | Ear | AST | Compound, predominantly | Sheets and nests of epithelioid cells with abundant | 9.5 | 2/mm2, | − | [symbol] |
| | | | | dermal, with congenital | amphophilic cytoplasm, well-defined | | superficial | | |
| | | | | nevus-like pattern | cytoplasmic borders and vesicular nuclei | | | | |
| | | | | | exhibiting moderate pleomorphism. Scattered | | | | |
| | | | | | giant/multinucleate tumor cells. | | | | |
| [symbol] | [symbol] | Post- | AST | Compound, predominantly | Sheets and nests of large epithelioid cells with | 4.0 | Absent | +++ | [symbol] |
| | | auricular | | dermal | abundant amphophilic cytoplasm, well- | | | | |
| | | | | | defined cytoplasmic borders and very | | | | |
| | | | | | pleomorphic round/oval vesicular nuclei | | | | |
| | | | | | with conspicuous nucleoli. | | | | |
| | | | | | Scattered giant/multinucleate tumor cells. | | | | |
| [symbol] | [symbol] | Upper | AST | Dermal | Sheets of large epithelioid cells with | 2.2 | 1/mm2, | + | [symbol] |
| | | back | | | amphophilic cytoplasm, well-defined | | superficial | | |
| | | | | | cytoplasmic borders and moderately | | | | |
| | | | | | pleomorphic vesicular nuclei with large | | | | |
| | | | | | nucleoli. | | | | |
| [symbol] | [symbol] | Upper | AST | Dermal | Sheets and nests of large epithelioid cells with | 2.6 | 1/mm2, | ++ | [symbol] |
| | | back | | | abundant amphophilic cytoplasm, well- | | superficial | | |
| | | | | | defined cytoplasmic borders and mildly | | | | |
| | | | | | pleomorphic round/oval vesicular nuclei. | | | | |
| | | | | | An occasional giant/multinucleate | | | | |
| | | | | | tumor cell. | | | | |
| [symbol] | [symbol] | Upper | AST | Dermal | Sheets of medium-sized epithelioid, oval and | 5.0 | Absent | − | [symbol] |
| | | back | | | spindle cells with amphophilic cytoplasm, | | | | |
| | | | | | well-defined cytoplasmic borders and mildly | | | | |
| | | | | | pleomorphic vesicular nuclei with small | | | | |
| | | | | | nucleoli. | | | | |
| [symbol] | [symbol] | n.a. | AST | Compound, predominantly | Sheets of medium-sized epithelioid, oval and | 2.4 | Absent | − | [symbol] |
| | | | | dermal | spindle cells with amphophilic cytoplasm, | | | | |
| | | | | | well-defined cytoplasmic borders and mildly | | | | |
| | | | | | pleomorphic vesicular nuclei with small | | | | |
| | | | | | nucleoli. Scattered giant/multinucleate | | | | |
| | | | | | tumor cells. | | | | |
| [symbol] | [symbol] | Arm | AST | Dermal | Spindle and epithelioid melanocytes arranged in | 3.1 | 2/mm2, | − | [symbol] |
| | | | | | nests and cords. The cells contain abundant | | superficial | | |
| | | | | | amphophilic cytoplasm and mildly pleomorphic, | | | | |
| | | | | | vesicular nuclei | | | | |
| [symbol] | [symbol] | Ear | AST, | Compound | Wedge-shaped tumor composed of epithelioid and | 2.15 | 1/mm2, | − | [symbol] |
| | | | recurrent | | spindle-shaped cells exhibiting mild cytologic | | deep | | |
| | | | | | atypia | | | | |
| [symbol] | [symbol] | Arm | AST | Dermal | Rounded superficial dermal tumor composed of | 0.6 | 0 | − | [symbol] |
| | | | | | plump epithelioid cells | | | | |
| [symbol] | [symbol] | Cheek | AST | Dermal | Trabeculae and sheets of moderalty atypical | 5.6 | 2/mm2; | ++ | [symbol] |
| | | | | | epithelioid cells with abundant cytoplasm | | deep | | |
| | | | | | and conspicuous nucleoli | | | | |
| [symbol] | [symbol] | Ankle | AST | Dermal | Fascicles and sheets of spindle-shaped and oval | 5.5 | 4/mm2, | − | [symbol] |
| | | | | | melanocytes exhibiting moderate nuclear | | superficial | | |
| | | | | | pleomorphism and large nucleoli | | | | |
| [symbol] | [symbol] | Thigh | AST | Compound, predominantly | Sheets of epithelioid cells with abundant | 2.3 | 2/mm2, | + | [symbol] |
| | | | | dermal | amphophilic cytoplasm, well-defined cytoplasmic | | superficial | | |
| | | | | | borders and round/oval vesicular | | | | |
| | | | | | nuclei with conspicuous nucleoli; strands | | | | |
| | | | | | of dense collagen separate the tumor cells into | | | | |
| | | | | | islands of varying size; at the periphery, tumor | | | | |
| | | | | | cells merge with adjacent dermal collagen in an | | | | |
| | | | | | infiltrating pattern | | | | |
| [symbol] | [symbol] | Thumb | AST | Dermal | Spindle and occasional epithelioid cells with small | 2.8 | 4/mm2, | − | [symbol] |
| | | | | | to moderate amounts of amphophilic cytoplasm. | | superficial | | |
| | | | | | Associated epidermal hyperplasia. Cells merge | | | | |
| | | | | | in an infiltrative fashion with dermal collagen | | | | |
| | | | | | at periphery of tumor. | | | | |
| [symbol] | [symbol] | Elbow | AST, | Compound | Ulcerated tumor. Predominantly spindel cells | 3.0 | 1/mm2, | ++ | [symbol] |
| | | | polypoid | | in nests and sheets. Cells contain relatively | | superficial | | |
| | | | | | uniform oval/spindle-shaped nuclei and small | | | | |
| | | | | | to moderate amounts of cytoplasm, with ill- | | | | |
| | | | | | defined cytoplasmic borders. | | | | |
| [symbol] | 12 | Dorsum | AST | Compound | Spindle and epithelioid cells arranged in nests | 5.7 | Absent | − | [symbol] |
| | | of foot | | | and sheets separated by bundles of dermal | | | | |
| | | | | | collagen. Cells contain round to oval nuclei | | | | |
| | | | | | exhibiting little pleomorphism. | | | | |
| [symbol] | 2 | Pinna | AST | Dermal | Sheets of spindle and a few epithelioid cells | 6.5 | 2/mm2, | + | [symbol] |
| | | | | | containing relatively uniform oval/spindle- | | superficial | | |
| | | | | | shaped nuclei and small to moderate amounts | | | | |
| | | | | | of amphophilic cytoplasm | | | | |
| [symbol] | 23 | Buttock | AST | Dermal | Clusters of small epithelioid cells with small | 5.0 | Absent | + | [symbol] |
| | | | | | amounts of amphophilic cytoplasm and round- | | | | |
| | | | | | oval nuclei with vesicular, clumped and cleared | | | | |
| | | | | | nuclear chromatin, and conspicuous nucleoli, | | | | |
| | | | | | often irregularly-shaped | | | | |
| [symbol] | 9 | Forearm | AST | Compound | Relatively uniform spindel and epithelioid cells | 8.0 | 3/mm2 | + | [symbol] |
| | | | | | arranged in nests and fascicles, separated by | | | | |
| | | | | | collagenous septa. Associated epidermal | | | | |
| | | | | | hyperplasia and hypergranulosis. | | | | |
| [symbol] | n.a. | n.a. | AST | Compound | Epithelioid and spindle cells arranged in nests. | 2.0 | 1/mm2, | − | [symbol] |
| | | | | | Cells contain moderate amounts of eosinophilic | | superficial | | |
| | | | | | cytoplasm and moderately pleomorphic nuclei | | | | |
| | | | | | with conspicuous nucleoli. Variable amounts | | | | |
| | | | | | of pigment, particularly in junctional component. | | | | |
| [symbol] | 14 | Ear | AST | Compound, polypoid | Variably pigmented, predominantly uniform | 5.7 | 2/mm2, | − | [symbol] |
| | | | | | spindle cells arranged in nests and sheets. | | superficial | | |
| | | | | | Associated extensive sclerosis of dermal component | | | | |
| | | | | | and epidermal hyperplasia. | | | | |
| [symbol] | 42 | Buttock | AST | Compound, predominantly | Sheets of variably pigmented epithelioid and | 0.85 | Absent | + | [symbol] |
| | | | | dermal | spindle cells with moderate amounts of cytoplasm | | | | |
| | | | | | and moderate nuclear pleomorphism | | | | |
| [symbol] | 12 | Lateral | AST | Compound | Sheets and nests of variably pigmented epithelioid | 4.5 | 2/mm2, | + | [symbol] |
| | | ankle | | | cells with highly pleomorphic round-oval nuclei | | | | |
| [symbol] | 8 | Leg | AST | Predominantly dermal | Large epithelioid and spindle-shaped cells with | 5.5 | 1/mm2, | ++ | [symbol] |
| | | | | | abundant eosinophilic cytoplasm, indistinct | | superficial | | |
| | | | | | cytoplasmic borders and moderately pleomorphic | | | | |
| | | | | | round-oval vesicular nuclei with prominent | | | | |
| | | | | | nucleoli | | | | |
| [symbol] | 10 | Knee | AST | Dermal | Spindle and epithelioid melanocytes arranged in | 2.9 | 3/mm2, | + | [symbol] |
| | | | | | fascicles separated by collagenous septa. The | | | | |
| | | | | | cells contain abundant amphophilic cytoplasm with | | | | |
| | | | | | well defined cytoplasmic borders, and their nuclei | | | | |
| | | | | | are highly pleomorphic, with large and | | | | |
| | | | | | polymorphic nucleoli | | | | |
| [symbol] | 10 | Knee | AST | Compound | Oval and spindle-shaped melanocytes with | 2.3 | 2/mm2, | + | [symbol] |
| | | | | | moderate amounts of amphophilic cytoplasm | | | | |
| | | | | | arranged in nests and trabeculae. Nuclei are | | | | |
| | | | | | vesicular and relatively uniform in size and shape. | | | | |
| [symbol] | 24 | n.a. | AST | Compound | Epithelioid and oval cells with abundant amphophilic | 3.3 | 3/mm2, | ++ | [symbol] |
| | | | | | cytoplasm and round/oval, moderately pleomorphic | | | | |
| | | | | | nuclei with conspicuous nucleoli | | | | |
| [symbol] | 11 | Upper | AST | Compound | Epithelioid cells with abundant amphophilic | 2.0 | 1/mm2, | + | [symbol] |
| | | cheek | | | cytoplasm, well-defined cytoplasmic borders and | | superficial | | |
| | | | | | vesicular nuclei exhibiting moderate pleomorphism. | | | | |
| [symbol] | 24 | Leg | AST | Compound | Predominantly nests of focally pigmented epithelioid | 1.5 | 2/mm2, | ++ | [symbol] |
| | | | | | cells with abundant amphophilic cytoplasm, well- | | superficial | | |
| | | | | | defined cytoplasmic borders and vesicular nuclei | | | | |
| | | | | | exhibiting moderate pleomorphism and very | | | | |
| | | | | | conspicuous nucleoli. Scattered giant/multinucleate | | | | |
| | | | | | tumor cells. | | | | |
| [symbol] | 12 | Chin | AST | Dermal | Epithelioid and oval cells with abundant amphophilic | 1.8 | 1/mm2, | + | [symbol] |
| | | | | | cytoplasm, well-defined cytoplasmic borders and | | superficial | | |
| | | | | | vesicular nuclei exhibiting moderate pleomorphism. | | | | |
| | | | | | Scattered giant/multinucleate tumor cells. | | | | |

AST = atypical Spitz tumor;
congenital nevus-like pattern = extension along adnexal structures,
n.a., not available,
wt, wildtype [symbol]
indicates data missing or illegible when filed

In the evaluation set, we assessed expression of BAP1 by immunohistochemistry (IHC) to determine its specificity and sensitivity. IHC was performed with an automated IHC system (Ventana BenchMark XT, Ventana Medical Systems, Inc., Tucson, Ariz.) using an alkaline phosphatase method and a red chromogen, according to the manufacturer's instructions. Briefly, following deparaffinization of paraffin tissue sections and heat-induced antigen retrieval, the sections were incubated with BAP1 antibody (clone C-4, 1:50 dilution, Santa Cruz Biotechnology, Inc., Santa Cruz, Calif.) for 1 hour. A subsequent amplification step was followed by incubation with Hematoxylin II counterstain for 4 minutes and then with blueing reagent for 4 minutes. Nuclei of keratinocytes of the epidermis and appendages, fibroblasts and lymphocytes served as internal controls for BAP1 expression. Tumors were scored as positive or negative depending on whether or not their nuclei stained with BAP 1.

DNA extraction and Sanger Sequencing

Tumor and non-tumor areas were separately microdissected from sections of archival paraffin-embedded tissue using a dissection microscope. DNA was extracted and purified with a QIAamp DNA FFPE Tissue Kit (Qiagen, Hilden, Germany) according to the manufacturer's instructions. Mutations in BAP1 (all exons), BRAF (exon 15), and HRAS (exons 1 and 2) were determined by direct sequencing using previously described primers.^{3,16} The PCR reaction conditions were 0.25 mM dNTPs, 0.4. BSA (New England Biolabs), 1 U Hotstar Taq (Qiagen), 1× Hotstar Taq buffer (Qiagen) and 0.4 μM primer. PCR consisted of 35 cycles of 95° C. (45 s), 57° C. (45 s) and 72° C. (45 s) after initial denaturation at 95° C. for 5 min. PCR reaction products were purified with the QIAquick PCR Purification kit (Qiagen) and then used as templates for sequencing in both directions using Big Dye v3.1 (Applied Biosystems). Dye terminators were removed using the CleanSEQ kit (Agencourt Biosciences), and subsequent products were run on the ABI PRISM 3730x1 (Applied Biosystems). Mutations were identified by using Sequencher 5.0 software (Gene Codes Corporation, Ann Arbor, Mich.) and only considered when variants were called in reads in both directions. Normal DNA was sequenced from the adjacent non-tumor tissue to determine whether the mutations were somatically acquired or germline.

Array-based Comparative Genomic Hybridization

BAP1-negative cases in which sufficient DNA was available were analyzed using array-based comparative genomic hybridization (CGH). DNA was labeled using the Bioprime Array CGH Genomic Labeling Kit according to the manufacturer's instructions (Invitrogen, Carlsberg, Calif.) as described previously.¹⁵ Briefly, 500 ng test and reference DNA (Promega, Madison, Wis.) were differentially labeled with dCTP-Cy5 and dCTP-Cy3, respectively (GE Healthcare, Piscataway, N.J.). Genome-wide analysis of DNA copy number changes was conducted using an oligonucleotide array containing 60,000 probes according to the manufacturer's protocol version 6.0 (Agilent, Santa Clara, Calif.). Slides were scanned using Agilent's microarray scanner G2505B and analyzed using Agilent Feature Extraction and DNA Workbench software 6.5.018.

### Results

Accuracy of Immunohistochemistry to Determine BAP1 Status

As negative controls (cases with functional loss of BAP1) we used the 42 epithelioid melanocytic tumors from BAP1 germline mutation carriers. Of these, 33 (79%) tumors showed bi-allelic loss of BAP1 (inactivating germline mutation combined with either somatic deletion, loss of heterozygosity, or mutation of the remaining wild type allele).¹⁶ All 33 cases (100%) with bi-allelic loss of BAP1 were IHC-negative. The remaining 9 (21%) of these 42 cases had a very similar histological appearance, but no detectable somatically acquired alteration of BAP1 (no somatic deletion, loss of heterozygosity, or mutation). All of these 9 cases also showed loss of BAP1 IHC expression, indicating that the wild type BAP1 allele had been silenced in a way not detected by our analysis. Internal controls consisting of the nuclei of non-tumor cells (keratinocytes and lymphocytes) confirmed that the immunohistochemistry procedure was working (FIG. 11E).

Our positive control set consisted of 29 common acquired melanocytic nevi and 17 conventional Spitz nevi, in which we did not find any BAP1 mutations in our previous study¹⁶. All of these cases showed strong nuclear BAP1 staining These results indicate that BAP1 immunohistochemistry may be a convenient way of assessing the functional status of BAP1 compared to array CGH or DNA sequencing.

Loss of BAP1 Expression by Immunohistochemistry Identifies a Morphologically Distinct Subset of Tumors

The 32 ASTs selected for this study showed histologic features overlapping those of Spitz nevus and melanoma. Concern for malignancy was raised by the presence of varying combinations of atypical features such as architectural asymmetry, increased cellularity, nuclear pleomorphism, nucleolar prominence and detectable mitotic figures (Table 6).

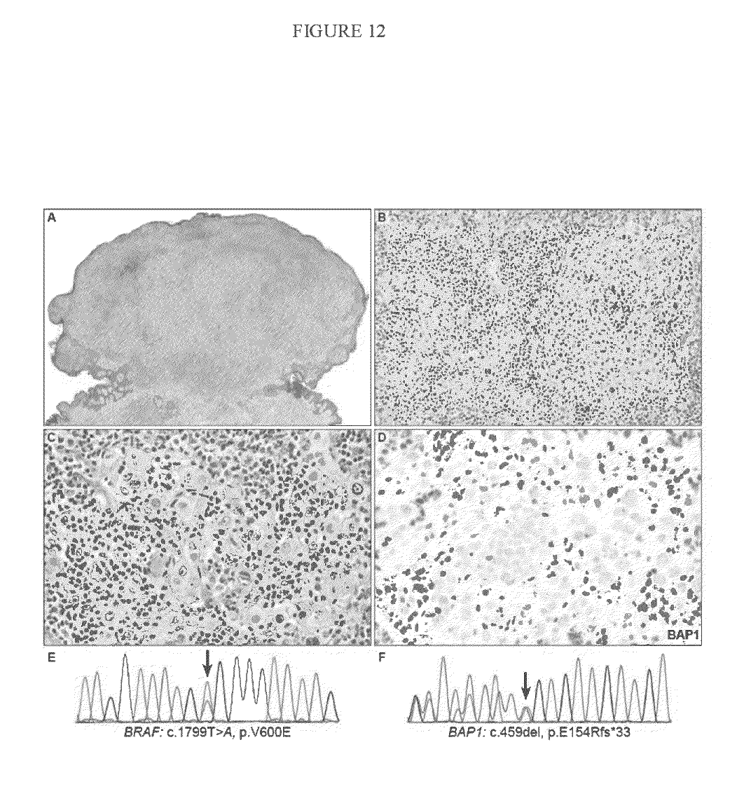

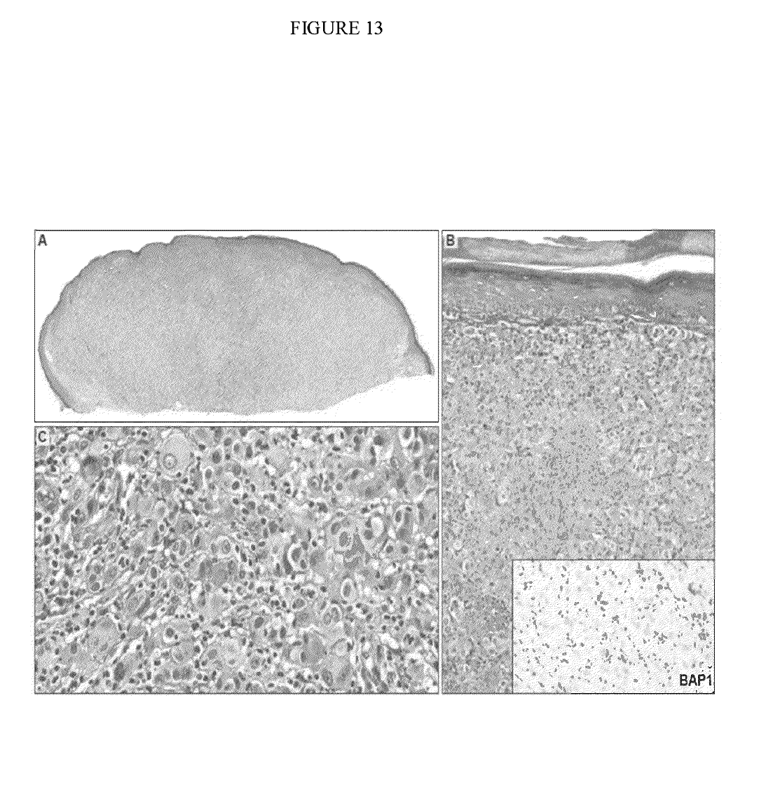

BAP1 immunohistochemistry was negative in 9 (28%) cases. The BAP1-negative tumors were predominantly or exclusively intradermal, while 11 of 23 (48%) of BAP1-positive tumors had a significant junctional component. Additional features shared by BAP 1-negative tumors were the predominance of plump epithelioid cells with moderate to large amounts of amphophilic cytoplasm and very well-demarcated cytoplasmic borders (FIGS. 12B, 13B, 14B, 15C, 16B). Their nuclei were round or oval in shape, and exhibited moderate pleomorphism with vesicular chromatin and often conspicuous nucleoli. There were also scattered giant cells, some of which were multinucleated (FIGS. 12C, 13C, 15C). In 3 of the 9 BAP1-negative cases (33%), significant numbers of tumor-infiltrating lymphocytes (TILS) were identified, resulting in a histologic appearance resembling that seen in so-called “halo Spitz nevus” (FIGS. 12, 13). Occasionally, aggregates of tumor cells were separated by delicate bands of collagen but prominent fibrosis or sclerosis was not a feature.

Several statistically significant differences between BAP1-negative and BAP1-positive tumors were observed. BAP1-negative neoplasms were located more frequently on the trunk and less often on the limbs, and were less mitotically active. They more often demonstrated sheet-like growth, contained amphophilic cytoplasm with well-defined cytoplasmic borders and marked tumor-infiltrating lymphocytes, and were less often composed of spindle-shaped or oval cells. Nuclear chromatin was more commonly vesicular, and binucleation and multinucleation were more frequently seen. While these differences were statistically significant, many of the cytologic features seen in the BAP1-negative tumors were also identified to varying degrees in some BAP1-positive tumors (Table 7).

TABLE 7
Comparison of clinico-pathologic features between BAP1− and BAP1+ tumors

| | | BAP1− (n = 9) | | BAP1+ (n = 23) | | |
|---|---|---|---|---|---|---|
| | | Mean/ | | Mean/ | | |
| Feature | | Median | Range | Median | Range | p value |
| Age (years) | | 29/21 | 12-59 | 18/12 | 2-52 | NS** |
| Breslow thickness (mm) | | 4.2/4 | 2.2-9.5 | 3.6/3 | 0.6-6.5 | NS** |
| Mitotic rate (per mm²) | | 0 | 0-2 | 2 | 0-4 | 0.01** |
| Feature | Level | N | % | N | % | p value |
| Anatomic site | Head/neck | 3 | 38 | 6 | 29 | 0.04† |
| | Trunk | 4 | 50 | 2 | 10 | |
| | Upper limbs | 1 | 13 | 5 | 24 | |
| | Lower limbs | 0 | 0 | 8 | 38 | |
| Architecture | Compound | 0 | 0 | 11 | 48 | 0.01† |
| | Dermal | 4 | 44 | 9 | 39 | |
| | Compound, predominantly | 5 | 56 | 3 | 13 | |
| | dermal | | | | | |
| Predominant | Sheet-like growth | 8 | 89 | 8 | 35 | 0.02* |
| growth pattern | Nested growth | 6 | 67 | 9 | 39 | NS* |
| Cellular shape | Epithelioid cells | 9 | 100 | 16 | 70 | NS* |
| | Spindle/Oval cells | 2 | 22 | 15 | 65 | 0.049* |
| Cytoplasmic | Cytoplasmic amphophilia | 9 | 100 | 9 | 39 | 0.002* |
| features | Well-defined cytoplasmic | 9 | 100 | 5 | 22 | <0.0001* |
| | borders | | | | | |
| Nuclear | Vesicular chromatin | 9 | 100 | 7 | 30 | 0.0008* |
| features | Moderate pleomorphism | 4 | 44 | 9 | 39 | NS* |
| | Marked pleomorphism | 2 | 22 | 2 | 9 | NS* |
| | Conspicuous nucleoli | 3 | 33 | 9 | 39 | NS* |
| | Binucleation/multinucleation | 7 | 78 | 2 | 9 | 0.0003* |
| Tumor- | Absent | 3 | 33 | 8 | 35 | 0.02† |
| infiltrating | Mild | 1 | 11 | 10 | 43 | |
| lymphocytes | Moderate | 2 | 22 | 5 | 22 | |
| | Marked | 3 | 33 | 0 | 0 | |

NS = not statistically significant (p > 0.05);
**Mann-Whitney test;
†Chi-squared test;
*Fisher exact test;

Mutations and Deletions of BAP1

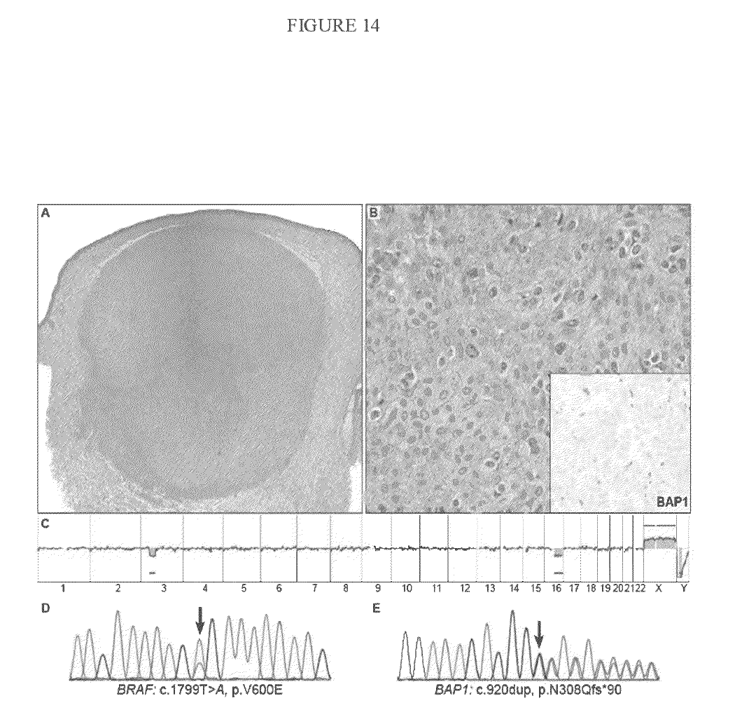

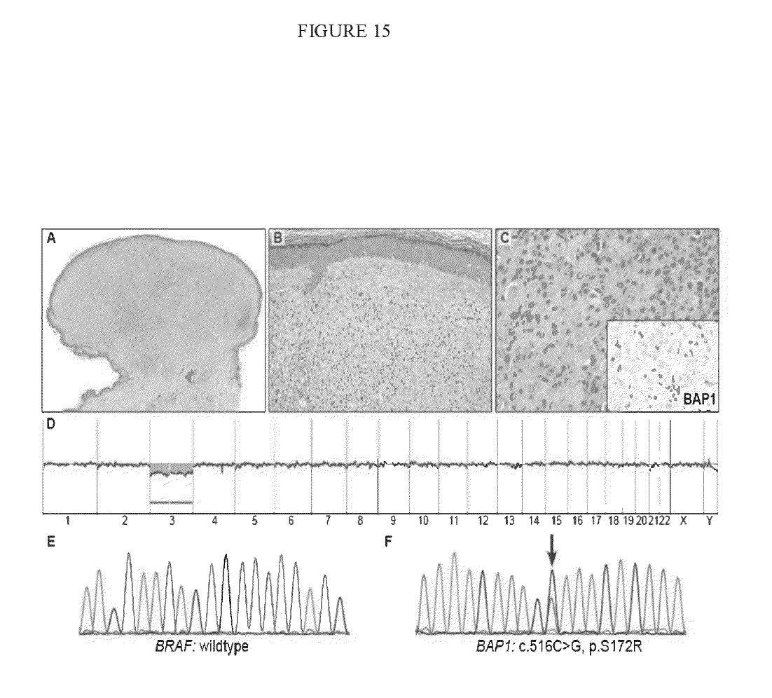

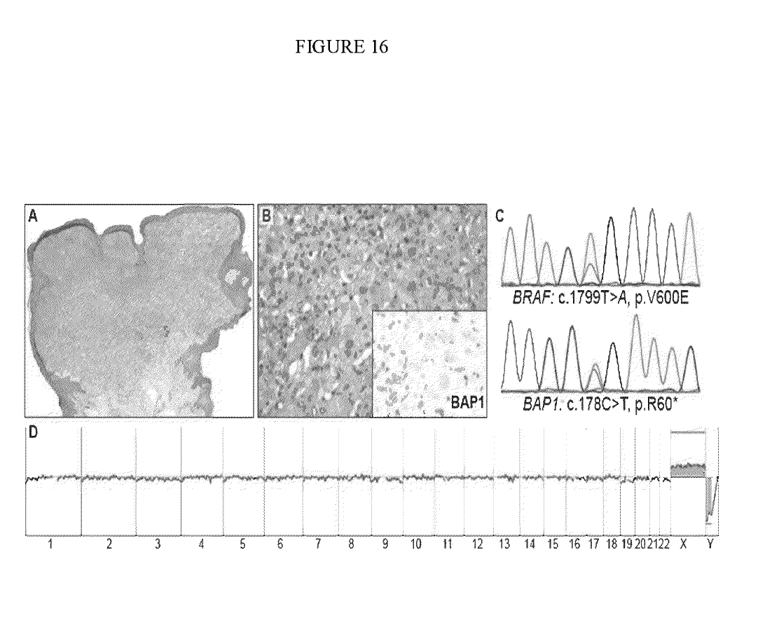

BAP1 was sequenced in all 9 BAP1-negative cases and somatically acquired mutations were found in 5 cases (56%, Table 3); two were frameshift mutations (FIGS. 12F, 14E), two were nonsense mutations (FIG. 16C), and one was a missense mutation (FIG. 15F). The 2 frameshift mutations and 2 nonsense mutations resulted in premature termination of the protein. No mutations were found in the remaining 4 BAP1-negative cases and in 20 of 23 BAP1-positive cases. In the remaining 3 BAP1-positive cases (case # 12, 14, and 15 in Table 6), BAP1 could not be fully sequenced. Array CGH was performed in 4 of the cases with negative BAP1 immunohistochemistry. In one case which had a BAP1 frameshift mutation there was focal loss of the BAP1 locus at 3p21 (FIG. 14C). Another case which bore a BAP1 missense mutation (FIG. 15F) was characterized by loss of the entire chromosome 3 (FIG. 15D). The remaining two cases showed no copy number changes of BAP1 or elsewhere in the genome (FIG. 16D).

TABLE 8
BRAF and BAP1 mutations in atypical Spitz tumors showing loss of BAP1 expression
by immunohistochemistry

| | BRAF | | BAP1 | | aCGH |
|---|---|---|---|---|---|
| Case # | DNA | Protein | DNA | Protein | 3p21 copy number status |
| 1 | c.1799T > A | p.V600E | c.459del | p.E154Rfs*33 | n.a. |
| 2 | c.1799T > A | p.V600E | wt | wt | n.a. |
| 3 | c.1799T > A | p.V600E | wt | wt | n.a. |
| 4 | c.1799T > A | p.V600E | wt | wt | n.a. |
| 5 | c.1799T > A | p.V600E | wt | wt | n.a. |
| 6 | c.1799T > A | p.V600E | c.178C > T | p.R60* | no loss |
| 7 | c.1799T > A | p.V600E | c.1768C > T | p.Q590* | no loss |
| 8 | c.1799T > A | p.V600E | c.920dup | p.N308Qfs*90 | loss |
| 9 | wt | wt | c.516C > G | p.S172R | loss |

n.a, not available,
wt, wildtype

Mutations of BRAF and HRAS

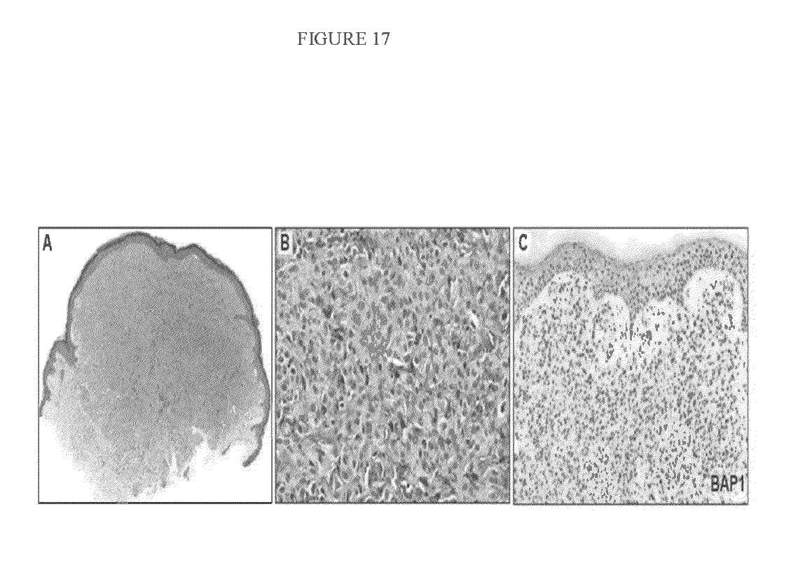

BRAF and HRAS were sequenced in 31 ASTs. Nine (29%) tumors carried a BRAF^{V600E} mutation (FIG. 12E, 14D, 16C), while no HRAS mutations were found (Table 6). There was a significant association between BRAF mutation status and loss of BAP1 by immunohistochemistry. Eight of the 9 (89%) BRAF^{V600E}-mutant tumors showed loss of BAP1 expression by IHC (Table 6, Fisher exact test: p<0.0001), suggesting that BRAF mutation and BAP1 loss characterizes a distinct subset of ASTs. Only one of the 9 BRAF mutated cases showed strong expression of BAP1 (FIG. 17). In this case, the tumor was cellular, and was composed of sheets of spindle-shaped, oval and epithelioid melanocytes with vesicular nuclei and amphophilic cytoplasm, but lacking well-defined cytoplasmic borders.

### Discussion

Traditional classification systems for melanocytic neoplasms rely on clinical and histologic characteristics to describe subtypes of nevi (e.g. acquired nevi, congenital nevi, Spitz nevi, blue nevi) and melanomas (e.g. superficial spreading melanomas, nodular melanomas, acral lentiginous melanomas, lentigo maligna melanomas). More recently, acquired mutations in oncogenes that lead to constitutive activation of critical signaling pathways have provided additional information useful for classification. These include BRAF mutations which are frequent in common acquired nevi¹⁰ and in melanomas from skin without chronic sun-induced damage⁸; KIT mutations in acral and mucosal melanoma and melanomas on skin with chronic sun-induced damage⁷; HRAS mutations in a subset of Spitz nevi³; and GNAQ and GNA11 mutations in blue nevi and uveal melanoma^{12,13}. Integration of underlying genetic aberrations and clinicopathologic features can lead to refined ways to classify melanocytic neoplasms and improve clinical relevance by incorporating information that can guide selection of targeted therapeutic agents.¹⁴

Here, we shed further light on the heterogeneous group of ASTs by demonstrating that they appear to be comprised of biologically distinct entities. In the present study, we have identified a histologic distinct subset of ASTs characterized by BRAF^{V600E} mutations and loss of BAP1 expression. In all cases, BAP1 loss was somatically acquired without evidence of a pre-existing germline mutation. These results demonstrate that melanocytic neoplasms with BAP1 alterations can occur outside of the previously described tumor predisposition syndrome caused by germline BAP1 mutations. We have also shown that BAP1 status can be reliably identified by immunohistochemistry, which will make BAP1 immunohistochemistry a useful tool for subtyping melanocytic neoplasms. Our observation that a greater number of tumors exhibited loss of BAP1 protein expression by immunohistochemistry than BAP1 deletions or mutations indicates that BAP1 may become functionally inactivated by means other than deletions or mutations in the coding region; for example, epigenetic changes leading to silencing of the BAP1 gene or other, yet to be identified, alterations that prevent BAP1 expression. Detailed in vitro and in vivo studies are needed to investigate the correlation between nuclear BAP1 protein expression (assessed by immunohistochemistry) and BAP1 functional activity.

The sporadic BRAF^{V600E}/BAP1^{neg.} tumors exhibited a typical epithelioid histomorphology that was previously seen in the BAP1-negative melanocytic skin tumors in patients with BAP1 germline mutations.¹⁶ The cytologic appearances of BRAF^{V600E}/BAP1^{neg.} tumors (plump epithelioid cells with amphophilic cytoplasm and very well-demarcated cytoplasmic borders; moderately pleomorphic round/oval nuclei with vesicular chromatin and variably conspicuous nucleoli; and multinucleate/giant cells) were distinctive, but were not absolutely specific, as some features were also identified to varying degrees in some BAP 1-positive tumors. Similarly, architectural features and stromal alterations absolutely specific to BAP1-negative tumors were not identified. TILs were prominent in many BAP1-negative tumors, but this was also not a specific diagnostic feature.

We previously reported that another subset of ASTs is characterized by HRAS mutations, copy number increases of chromosome 11p, and distinctive microscopic features.^{3,11} These tumors had similar cytologic features to those described here and were also predominantly intradermal. In contrast to the BRAF^{V600E}/BAP1^{neg.} tumors, the HRAS mutant neoplasms were associated with marked desmoplasia and did not show densely cellular aggregates. These results indicate that BRAF^{V600E} mutations and loss of BAP1 defines an additional genetic/morphologic subset of epithelioid ASTs (8/32, 25%). Thus, to date, spitzoid tumors are comprised of three distinguishable categories, HRAS mutant, BRAF^{V600E} /BAP1 mutant, and a (probably still heterogeneous) category of tumors with as yet unknown genetic characteristics.

Unfortunately, we did not have follow-up information on the tumors analyzed in our study so that we cannot address the important question of whether BAP1 or BRAF status provides any prognostic information about the tumors. In our experience, patients with ASTs almost invariably have an uneventful follow-up.⁴ Only a small minority develop widespread metastasis, raising the possibility that they were melanoma from the outset. Future studies are necessary to determine which, if any, genetic or morphologic criteria identified in this study can help in the risk assessment of these tumors.

In conclusion, we have described a distinct subset of ASTs characterized by loss of BAP1 expression, BRAF^{V600E} mutations, and some distinctive histologic features. Our findings establish a platform for future studies to investigate the prognostic significance of genetically-defined subsets of ASTs.

### REFERENCES

1. Barnhill R L. The Spitzoid lesion: rethinking Spitz tumors, atypical variants, ‘Spitzoid melanoma’ and risk assessment. Mod Pathol. 2006;19 Suppl 2:S21-33.
2. Barnhill R L, Argenyi Z B, From L, et al. Atypical Spitz nevi/tumors: lack of consensus for diagnosis, discrimination from melanoma, and prediction of outcome. Hum Pathol. 1999;30:513-520.
3. Bastian B C, LeBoit P E, Pinkel D. Mutations and copy number increase of HRAS in Spitz nevi with distinctive histopathological features. Am J Pathol. 2000;157:967-972.
4. Busam K J, Murali R, Pulitzer M, et al. Atypical spitzoid melanocytic tumors with positive sentinel lymph nodes in children and teenagers, and comparison with histologically unambiguous and lethal melanomas. Am J Surg Pathol. 2009;33:1386-1395.
5. Cerroni L, Barnhill R, Elder D, et al. Melanocytic tumors of uncertain malignant potential: results of a tutorial held at the XXIX Symposium of the International Society of Dermatopathology in Graz, October 2008. Am J Surg Pathol. 2010;34:314-326.
6. Crotty K A. Spitz naevus: histological features and distinction from malignant melanoma. Australasian Journal of Dermatology. 1997;38 Suppl 1:S49-53.
7. Curtin J A, Busam K, Pinkel D, et al. Somatic activation of KIT in distinct subtypes of melanoma. J Clin Oncol. 2006;24:4340-4346.
8. Curtin J A, Fridlyand J, Kageshita T, et al. Distinct sets of genetic alterations in melanoma. N Engl J Med. 2005;353:2135-2147.
9. Palmedo G, Hantschke M, Rutten A, et al. The T1796A mutation of the BRAF gene is absent in Spitz nevi. J Cutan Pathol. 2004;31:266-270.
10. Pollock P M, Harper U L, Hansen K S, et al. High frequency of BRAF mutations in nevi. Nat Genet. 2003;33:19-20.
11. van Engen-van Grunsven A C, van Dijk M C, Ruiter D J, et al. HRAS-mutated Spitz tumors: A subtype of Spitz tumors with distinct features. Am J Surg Pathol. 2010;34:1436-1441.
12. Van Raamsdonk C D, Bezrookove V, Green G, et al. Frequent somatic mutations of GNAQ in uveal melanoma and blue naevi. Nature. 2009;457:599-602.
13. Van Raamsdonk C D, Griewank K G, Crosby M B, et al. Mutations in GNA11 in uveal melanoma. N Engl J Med. 2010;363:2191-2199.
14. Whiteman D C, Pavan W J, Bastian B C. The melanomas: a synthesis of epidemiological, clinical, histopathological, genetic, and biological aspects, supporting distinct subtypes, causal pathways, and cells of origin. Pigment Cell Melanoma Res. 2011;24:879-897.
15. Wiesner T, Obenauf A C, Cota C, et al. Alterations of the cell-cycle inhibitors p27(KIP1) and p16(INK4a) are frequent in blastic plasmacytoid dendritic cell neoplasms. J Invest Dermatol. 2010;130:1152-1157.
16. Wiesner T, Obenauf A C, Murali R, et al. Germline mutations in BAP1 predispose to melanocytic tumors. Nat Genet. 2011;43:1018-1021.

### EXAMPLE 3

### Towards an Improved Definition of the Tumor Spectrum Associated with BAP1 Germline Mutations

BAP1 is a tumor suppressor gene located on chromosome 3p21, a region which is deleted in several cancers, including mesothelioma, cutaneous and uveal melanoma, and cancers of the lung and breast. Somatic BAP1 mutations are common in uveal melanoma(1) and mesothelioma(2). We recently found and reported that germline mutations in BAP1 predispose to multiple clinically and pathologically distinctive epithelioid melanocytic tumors, and also to uveal and cutaneous melanomas(see Example 1 above, 3). Subsequent to our work, two other studies showed that BAP1 germline mutations predispose to other types of cancer—Testa and colleagues(4) reported that BAP1 germline mutations predispose to malignant mesothelioma and uveal melanoma, while Abdel-Rahman et al(5) suggested that BAP1 germline mutations may be also associated with lung adenocarcinoma, meningioma, and other cancers. Because of the involvement of BAP1 in various biologic processes, including chromatin dynamics(6), DNA damage response(7,8), and regulation of the cell cycle and cell growth(9), it was suggested that different germline BAP1 mutations might cause dissimilar cancer related syndromes and predispose to different tumor types(10), a proposal supported by the occurrence of distinct sets of tumors in patients in the aforementioned reports(3-5). In this report, we provide evidence of overlap in the tumor phenotypes that may be seen in BAP1 mutation carriers and discuss the tumor spectrum associated with BAP1 germline mutations.

Family 1

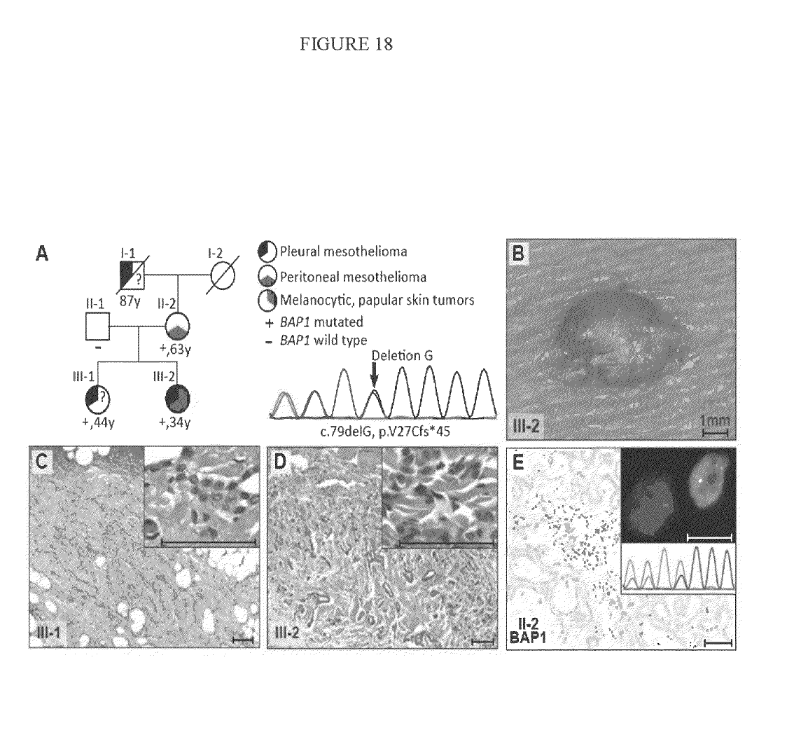

We identified a family in which mesotheliomas were inherited in an autosomal dominant pattern over three generations (FIG. 18A, age in years at diagnosis is indicated below the 3 symbols). The family was of European descent and none of the family members had a history of exposure to asbestos or erionite. Three of four affected family members with mesothelioma were long-term survivors. Subject II-2 was in complete remission after resection of a peritoneal mesothelioma 6 years ago, subject III-1 was in complete remission after resection of a pleural mesothelioma 2 years ago, and subject III-2 had a slowly progressing malignant peritoneal effusion 8 years after resection and chemotherapy of pleural and peritoneal mesothelioma. Only subject I-1 died of a pleural mesothelioma within 2 years. The indolent clinical course contrasted with the comparatively rapid progression of sporadic and asbestos-associated mesotheliomas, which are characterized by a median survival of less than 18 months from diagnosis(11).

We were able to examine the skin of two family members (i.e. II-2 and III-2) for the presence of the typical BAP1-associated melanocytic skin tumors, as described previously(3). Subject III-2 exhibited the characteristic melanocytic tumors, which presented clinically as inconspicuous, skin-colored to reddish-brown, dome-shaped papules (FIG. 18B). Subject II-2 lacked cutaneous melanocytic tumors. We sequenced DNA from 3 affected (II-2, III-1, and III-2) and one unaffected (II-1) family members for BAP1 germline mutations using the previously described primers and PCR conditions for Sanger sequencing(3, see Example 1 ,Table 5). We identified a BAP1 germline mutation (c.79delG,p.V27Cfs*45) in all affected family members, but not in the unaffected individual (FIG. 18A). In addition, we performed ‘whole-exome sequencing’ in subjects III-1 and III-2, however, we did not find mutations in any other known cancer-related gene.

We also investigated BAP1 status in mesotheliomas from subjects II-2, III-1, and III-2. Histologically, the malignant mesotheliomas were composed of cords and tubules of malignant cells (FIG. 18C, 18D; hematoxylin-eosin-stain). In all three mesotheliomas, interphase fluorescence in situ hybridization (FISH) did not show loss of the BAP1 region, but in two of the three mesotheliomas the sequencing electropherogram showed reduced intensity of the wild-type sequence, suggesting that the neoplastic cells had lost the wild-type BAP1 allele through copy number-neutral mechanisms. Immunohistochemistry showed loss of nuclear BAP 1 staining in all 3 cases of mesothelioma, but positive staining in intermingled lymphocytes (FIG. 18E).

Family 2

Further evidence of phenotypic overlap that may be seen in BAP1 mutation carriers is provided by the very recent diagnosis of peritoneal mesothelioma in a member (subject II-6 of family 2 in FIG. 1a in ref 3 and in Example 1) of one of our two previously published families with multiple melanocytic tumors, including epithelioid nevi, cutaneous melanoma and uveal melanoma. This patient had no known history of occupational or environmental exposure to asbestos or erionite.

Family 3

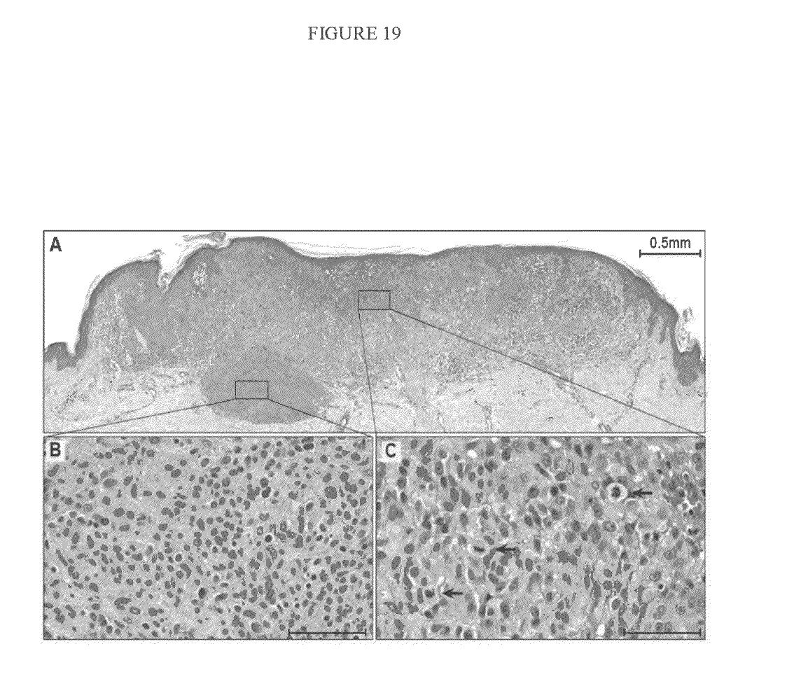

Cutaneous melanoma had developed in one of our two initially published families (family 2 in ref 3 and in Example 1) at the time of publication. Subsequently however, a member of the other family (subject II-2 of family 1 in Figure 1a in ref 3 and in Example 1) was also diagnosed with cutaneous melanoma. Intimately associated with the base of the melanoma, there was residual epithelioid nevus (FIG. 19A). The nevus component was composed of epithelioid cells and moderate nuclear pleomorphism, typical of the melanocytic skin tumors seen in patients with BAP1 germline mutations (FIG. 19B). The melanoma component (FIG. 19C) comprised large, pleomorphic melanocytes displaying vesicular nuclei, prominent nucleoli, and mitotic figures (arrows). The histologic appearances strongly suggested that the melanoma arose from the nevus. Both melanoma and nevus showed loss of BAP1 expression by immunohistochemistry (data not shown).

### Discussion

BAP1 is a tumor susceptibility gene, but the complete phenotypic spectrum of tumors in BAP1 mutation carriers remains to be accurately defined. Our novel data provide strong evidence that melanocytic tumors and mesotheliomas may co-occur in BAP1 germline mutation carriers. Interestingly, 3 of the 5 patients with mesothelioma (two of the four affected family members from the family 1, as well as the newly diagnosed patient from family 2) had peritoneal mesotheliomas. This contrasts with sporadic mesothelioma, which arises in the pleura in about 70% of cases(12). This is in keeping with previous reports about ‘mesothelioma villages’, describing autosomal dominant inheritance of mesothelioma in certain families in which the incidence of peritoneal mesothelioma to pleural mesothelioma was approximately 1:1 (13,14). Thus, the incidence of peritoneal mesotheliomas may be relatively increased in the setting of hereditary susceptibility.

The identification of cutaneous melanoma in two separate families suggests that it is also a BAP1-associated tumor. Our findings indicate that some of the typical epithelioid melanocytic skin tumors in BAP1 mutation carriers may progress to melanoma. Thus, in melanocytes of the skin, BAP1 may play a similar role to that of APC in familial adenomatous polyposis. Biallelic inactivation of APC causes multiple colorectal adenomas (akin to multiple epithelioid tumors in patients with biallelic loss of BAP1). The adenomas (like the epithelioid tumors) undergo malignant transformation at a low frequency and usually require other mutations to do so. We previously provided evidence that such additional mutations in the melanocytic skin tumors involve BRAF (3 and Example 1).

We identified 19 BAP1 germline mutation carriers in our three families. 16 (84.3%) carriers developed epitheloid nevi; five (26.3%) subjects were diagnosed with mesothelioma (2 peritoneal and 2 pleural, and 1 with both pleural and peritoneal); and cutaneous and uveal melanoma occurred in 4 (21.1%) and 2 (10.5%) subjects, respectively. Although we did not observe other cancers in our families, Testa et al(4) and Abdel-Rahman et al(5) reported occurrence in BAP1 mutation carriers of other tumors such as cancers of the ovary, breast, kidney, pancreas, prostate, lung, as well as meningioma. However, according to the COSMIC (Catalogue Of Somatic Mutations In Cancer; sanger.ac.uk/genetics/CGP/cosmic) database, 15 somatic BAP1 mutations in these cancers are very rare, i.e. in ovarian cancer (2%; 1 mutation in 58 cases), breast cancer (0% 1/251), pancreatic cancer (0%; 0/30), prostate cancer (0%; 0/58), and lung adenocarcinoma (1%; 2/322). The COSMIC database does not yet include BAP1 mutation data on renal cell cancer and meningioma. Whether these data imply a role for BAP1 in these tumors or whether BAP1 is inactivated by other means is presently unclear. Extensive genetic epidemiologic studies are required to determine if these tumors are part of the BAP1-related tumor susceptibility spectrum.

In summary, our novel data support the notion that BAP1-germline mutation carriers are predisposed to the development of melanocytic skin lesions, uveal melanoma, and mesothelioma with varying degrees of penetrance. The variation in the number of melanocytic tumors and in the incidence of mesothelioma among BAP1 mutation carriers may reflect differences in the genetic background of the individuals or differences in their exposure to external contributing factors such as solar ultraviolet radiation or asbestos. Whether BAP1 germline mutations also predispose to lung cancer and other cancer types as recently suggested (4,5) remains to be fully clarified. A more precise definition of the tumor susceptibility spectrum in patients with BAP1 germline mutations is important for accurate genetic counseling and the institution of appropriate surveillance programs for affected individuals.

### REFERENCES

1. Harbour J W, Onken M D, Roberson E D, et al: Frequent mutation of BAP1 in metastasizing uveal melanomas. Science 330:1410-3, 2010
2. Bott M, Brevet M, Taylor B S, et al: The nuclear deubiquitinase BAP1 is commonly inactivated by somatic mutations and 3p21.1 losses in malignant pleural mesothelioma. Nature Genetics 43:668-72, 2011
3. Wiesner T, Obenauf A C, Murali R, et al: Germline mutations in BAP1 predispose to melanocytic tumors. Nature Genetics 43:1018-21, 2011
4. Testa J R, Cheung M, Pei J, et al: Germline BAP1 mutations predispose to malignant mesothelioma. Nature Genetics 43:1022-5, 2011
5. Abdel-Rahman M H, Pilarski R, Cebulla C M, et al: Germline BAP1 mutation predisposes to uveal melanoma, lung adenocarcinoma, meningioma, and other cancers. Journal of Medical Genetics 48:856-9, 2011
6. Scheuermann J C, de Ayala Alonso A G, Oktaba K, et al: Histone H2A deubiquitinase activity of the Polycomb repressive complex PR-DUB. Nature 465:243-7, 2010
7. Stokes M P, Rush J, Macneill J, et al: Profiling of UV-induced ATM/ATR signaling pathways. Proc Natl Acad Sci U S A 104:19855-60, 2007
8. Matsuoka S, Ballif B A, Smogorzewska A, et al: ATM and ATR substrate analysis reveals extensive protein networks responsive to DNA damage. Science 316:1160-6, 2007
9. Jensen D E, Proctor M, Marquis S T, et al: BAP1: a novel ubiquitin hydrolase which binds to the BRCA1 RING finger and enhances BRCA1-mediated cell growth suppression. Oncogene 16:1097-112, 1998
10. Goldstein A M: Germline BAP1 mutations and tumor susceptibility. Nature Genetics 43:925-6, 2011
11. Yang H, Testa J R, Carbone M: Mesothelioma epidemiology, carcinogenesis, and pathogenesis. Current treatment options in oncology 9:147-57, 2008
12. Bridda A, Padoan I, Mencarelli R, et al: Peritoneal mesothelioma: a review. MedGenMed : Medscape general medicine 9:32, 2007
13. Carbone M, Emri S, Dogan A U, et al: A mesothelioma epidemic in Cappadocia: scientific developments and unexpected social outcomes. Nature Reviews Cancer 7:147-54, 2007
14. Roushdy-Hammady I, Siegel J, Emri S, et al: Genetic-susceptibility factor and malignant mesothelioma in the Cappadocian region of Turkey. Lancet 357:444-5, 2001
15. Forbes S A, Bindal N, Bamford S, et al: COSMIC: mining complete cancer genomes in the Catalogue of Somatic Mutations in Cancer. Nucleic Acids Res 39:D945-50, 2011

This invention may be embodied in other forms or carried out in other ways without departing from the spirit or essential characteristics thereof The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method of diagnosing or prognosing melanoma in a mammal comprising determining the presence of BAP1 protein and/or the presence of a mutation in the BAP1 gene that inhibits expression of BAP1 or function of the BAP1 protein, wherein the absence of BAP1 protein and/or the presence of said mutation indicates a diagnosis of melanoma or a prognosis of risk of melanoma in said mammal.

2. A method for characterizing and/or classifying a skin lesion from a subject comprising obtaining a sample of the skin lesion and determining the presence of BAP1 protein and/or the presence of a mutation in the BAP1 gene that inhibits expression of BAP1 or function of the BAP1 protein.

3. The method of claim 1 or 2 wherein the presence of BAP1 protein and/or the presence of a mutation in the BAP1 gene is determined in a biopsy or skin lesion sample.

4. The method of claim 1 or 2 wherein the presence of BAP1 protein is determined by binding of a BAP1 specific antibody.

5. The method of claim 1 or 2 wherein the presence of a germline or somatic mutation in the BAP1 gene is determined by BAP1 sequence analysis of DNA or RNA in normal cells or in skin lesion cells.

6. The method of claim 1 or 2 wherein the mammal or subject is a human.

7. A method to determine a susceptibility to melanocytic neoplasms in a mammal comprising testing a tissue or cellular sample from said mammal to ascertain the presence of a germline mutation in the BAP1 gene that inhibits expression of BAP1 or function of the protein encoded by BAP1, such mutation indicating a susceptibility to melanocytic neoplasms.

8. The method of claim 7 wherein the sample is isolated from prenatal or embryonic cells.

9. A method to determine a melanocytic nevus at high risk for malignant transformation comprising testing a sample of the nevus to ascertain the presence, in the BAP1 gene, of a mutation that affects expression of BAP1 or function of the protein encoded by BAP1, such mutation indicating a high risk for malignant transformation.

10. The method of claim 7 or 9 wherein the sample is DNA or RNA.

11. The method of claim 7 or 9 wherein the sample is protein.

12. The method of claim 7 or 9 wherein the presence of BAP1 protein is determined by binding of a BAP1 specific antibody.

13. The method of claim 7 or 9 wherein the presence of a germline or somatic mutation in the BAP1 gene is determined by BAP1 sequence analysis of DNA or RNA in normal cells or in skin lesion cells.

14. An assay or kit for determining predisposition to or risk of melanocytic neoplasms in a subject wherein BAP1 protein expression or activity and/or the sequence of the BAP1 gene or existence of BAP1 mutations causing altered BAP 1 protein expression or activity are determined.

15. The assay or kit of claim 14 comprising:
a) a composition comprising one or more BAP1 specific primer pair in an amount effective to permit amplification and sequencing of BAP1 nucleic acid sequence in said sample and a biologically compatible salt solution and/or a composition comprising at least one BAP1 specific antibody effective to detect BAP1 protein in said sample suitably labeled for direct or indirect detection;
b) positive and negative control nucleic acid sequences of the BAP/sequence, and/or positive control BAP 1 protein; and
c) an instructional material setting forth a protocol suitable for use in detecting and/or quantifying BAP1 mutant nucleic acid sequences and/or BAP1 protein.

16. The assay or kit of claim 15 wherein the one or more BAP1 specific primer pair is selected from Table 5 or SEQ ID NOS: 3-40.
